SEQUENCE LISTING (70 sequences)

SEQ ID NO: 1 - DNA, 20 bp, Artificial Sequence
  Synthetic nucleic acid sequence
attttcagtgtattgctttg

SEQ ID NO: 2 - DNA, 20 bp, Artificial Sequence
  Synthetic nucleic acid sequence
acatcaccccataaacaaat

SEQ ID NO: 3 - DNA, 20 bp, Artificial Sequence
  Synthetic nucleic acid sequence
agggtgaactcactggaacg

SEQ ID NO: 4 - DNA, 20 bp, Artificial Sequence
  Synthetic nucleic acid sequence
tctaaatcaccacgatcaaa

SEQ ID NO: 5 - DNA, 20 bp, Artificial Sequence
  Synthetic nucleic acid sequence
tttcccgtgggggtgtggct

SEQ ID NO: 6 - DNA, 20 bp, Artificial Sequence
  Synthetic nucleic acid sequence
aaactttcgtttattgctaa

SEQ ID NO: 7 - DNA, 20 bp, Artificial Sequence
  Synthetic nucleic acid sequence
aatcgtgtgaccgcggtggc

SEQ ID NO: 8 - DNA, 20 bp, Artificial Sequence
  Synthetic nucleic acid sequence
atctaaaacactctttacgc

SEQ ID NO: 9 - DNA, 20 bp, Artificial Sequence
  Synthetic nucleic acid sequence
actggagttttttacaactc

SEQ ID NO: 10 - DNA, 20 bp, Artificial Sequence
  Synthetic nucleic acid sequence
cacaaaatagactacgaaag

SEQ ID NO: 11 - DNA, 20 bp, Artificial Sequence
  Synthetic nucleic acid sequence
ggggtatctaatcccagttt

SEQ ID NO: 12 - DNA, 20 bp, Artificial Sequence
  Synthetic nucleic acid sequence
gatttaactgttgaggttta

SEQ ID NO: 13 - DNA, 20 bp, Artificial Sequence
  Synthetic nucleic acid sequence
gtcctttgagttttaagctg

SEQ ID NO: 14 - DNA, 20 bp, Artificial Sequence
  Synthetic nucleic acid sequence
acagaacaggctcctctaga

SEQ ID NO: 15 - DNA, 20 bp, Artificial Sequence
  Synthetic nucleic acid sequence
tatataggctgagcaagagg

SEQ ID NO: 16 - DNA, 20 bp, Artificial Sequence
  Synthetic nucleic acid sequence
tcttcagcaaaccctgatga

SEQ ID NO: 17 - DNA, 20 bp, Artificial Sequence
  Synthetic nucleic acid sequence
cccatttcttgccacctcat

SEQ ID NO: 18 - DNA, 20 bp, Artificial Sequence
  Synthetic nucleic acid sequence
tcgacccttaagtttcataa

SEQ ID NO: 19 - DNA, 20 bp, Artificial Sequence
  Synthetic nucleic acid sequence
tgaaacttaagggtcgaagg

SEQ ID NO: 20 - DNA, 20 bp, Artificial Sequence
  Synthetic nucleic acid sequence
gtatacttgaggagggtgac

SEQ ID NO: 21 - DNA, 20 bp, Artificial Sequence
  Synthetic nucleic acid sequence
ctttgtgttaagctacactc

SEQ ID NO: 22 - DNA, 20 bp, Artificial Sequence
  Synthetic nucleic acid sequence
aaggttgtctggtagtaagg

SEQ ID NO: 23 - DNA, 20 bp, Artificial Sequence
  Synthetic nucleic acid sequence
catttacccaaataaagtat

SEQ ID NO: 24 - DNA, 20 bp, Artificial Sequence
  Synthetic nucleic acid sequence
agtccttgctatattatgct

SEQ ID NO: 25 - DNA, 20 bp, Artificial Sequence
  Synthetic nucleic acid sequence
taactagaaataactttgca

SEQ ID NO: 26 - DNA, 20 bp, Artificial Sequence
  Synthetic nucleic acid sequence
cactattttgctacatagac

SEQ ID NO: 27 - DNA, 20 bp, Artificial Sequence
  Synthetic nucleic acid sequence
ctaccgagcctggtgatagc

SEQ ID NO: 28 - DNA, 20 bp, Artificial Sequence
  Synthetic nucleic acid sequence
aggggatttagagggttctg

SEQ ID NO: 29 - DNA, 20 bp, Artificial Sequence
  Synthetic nucleic acid sequence
ggaacagctctttggacact

SEQ ID NO: 30 - DNA, 20 bp, Artificial Sequence
  Synthetic nucleic acid sequence
ggctgcttttaggcctacta

SEQ ID NO: 31 - DNA, 20 bp, Artificial Sequence
  Synthetic nucleic acid sequence
tttgggattttttaggtagt

SEQ ID NO: 32 - DNA, 20 bp, Artificial Sequence
  Synthetic nucleic acid sequence
gattggtccaattgggtgtg

SEQ ID NO: 33 - DNA, 20 bp, Artificial Sequence
  Synthetic nucleic acid sequence
actaacattagttcttctat

SEQ ID NO: 34 - DNA, 20 bp, Artificial Sequence
  Synthetic nucleic acid sequence
tgatctgacgcaggcttatg

SEQ ID NO: 35 - DNA, 20 bp, Artificial Sequence
  Synthetic nucleic acid sequence
tgttggttgattgtagatat

SEQ ID NO: 36 - DNA, 20 bp, Artificial Sequence
  Synthetic nucleic acid sequence
cttatgagcatgcctgtgtt

SEQ ID NO: 37 - DNA, 20 bp, Artificial Sequence
  Synthetic nucleic acid sequence
gaaaggttaaaaaaagtaaa

SEQ ID NO: 38 - DNA, 20 bp, Artificial Sequence
  Synthetic nucleic acid sequence
gcaggcggtgcctctaatac

SEQ ID NO: 39 - DNA, 20 bp, Artificial Sequence
  Synthetic nucleic acid sequence
tttgcacggttagggtaccg

SEQ ID NO: 40 - DNA, 20 bp, Artificial Sequence
  Synthetic nucleic acid sequence
cctcgtggagccattcatac

SEQ ID NO: 41 - DNA, 20 bp, Artificial Sequence
  Synthetic nucleic acid sequence
cacgggcaggtcaatttcac

SEQ ID NO: 42 - DNA, 20 bp, Artificial Sequence
  Synthetic nucleic acid sequence
taataaattaaagctccata

SEQ ID NO: 43 - DNA, 20 bp, Artificial Sequence
  Synthetic nucleic acid sequence
ttaggacctgtgggtttgtt

SEQ ID NO: 44 - DNA, 20 bp, Artificial Sequence
  Synthetic nucleic acid sequence
tgcattaaaaatttcggttg

SEQ ID NO: 45 - DNA, 20 bp, Artificial Sequence
  Synthetic nucleic acid sequence
aagtcttagcatgtactgct

SEQ ID NO: 46 - DNA, 20 bp, Artificial Sequence
  Synthetic nucleic acid sequence
tgttccgttggtcaagttat

SEQ ID NO: 47 - DNA, 20 bp, Artificial Sequence
  Synthetic nucleic acid sequence
gttgatatggactctagaat

SEQ ID NO: 48 - DNA, 20 bp, Artificial Sequence
  Synthetic nucleic acid sequence
tacgacctcgatgttggatc

SEQ ID NO: 49 - DNA, 20 bp, Artificial Sequence
  Synthetic nucleic acid sequence
gatggtgcagccgctattaa

SEQ ID NO: 50 - DNA, 20 bp, Artificial Sequence
  Synthetic nucleic acid sequence
ggtctgaactcagatcacgt

SEQ ID NO: 51 - DNA, 20 bp, Artificial Sequence
  Synthetic nucleic acid sequence
tcttgtcctttcgtacaggg

SEQ ID NO: 52 - DNA, 20 bp, Artificial Sequence
  Synthetic nucleic acid sequence
tgagatgatatcatttacgg

SEQ ID NO: 53 - DNA, 20 bp, Artificial Sequence
  Synthetic nucleic acid sequence
cccacacccacccaagaaca

SEQ ID NO: 54 - DNA, 20 bp, Artificial Sequence
  Synthetic nucleic acid sequence
acttaaaactttacagtcag

SEQ ID NO: 55 - DNA, 20 bp, Artificial Sequence
  Synthetic nucleic acid sequence
aaacttgtggtagttggagc

SEQ ID NO: 56 - DNA, 20 bp, Artificial Sequence
  Synthetic nucleic acid sequence
acaaatattttaatacatga

SEQ ID NO: 57 - DNA, 60 bp, Artificial Sequence
  Synthetic nucleic acid sequence
  misc_feature (19)..(38): n may be a, g, c, or t.
taatacgactcactatagnnnnnnnnnnnnnnnnnnnngtttaagagctatgctggaaac

SEQ ID NO: 58 - DNA, 90 bp, Artificial Sequence
  Synthetic nucleic acid sequence
aaaaaaagcaccgactcggtgccactttttcaagttgataacggactagccttatttaaacttgctatgctgtttccagcatagctctta

SEQ ID NO: 59 - DNA, 18 bp, Artificial Sequence
  Synthetic nucleic acid sequence
taatacgactcactatag

SEQ ID NO: 60 - DNA, 22 bp, Artificial Sequence
  Synthetic nucleic acid sequence
aaaaaaagcaccgactcggtgc

SEQ ID NO: 61 - DNA, 131 bp, Artificial Sequence
  Synthetic nucleic acid sequence
  misc_feature (19)..(38): n may be a, g, c, or t.
taatacgactcactatagnnnnnnnnnnnnnnnnnnnngtttaagagctatgctggaaacagcatagcaagtttaaataaggctagtccgttatcaacttgaaaaagtggcaccgagtcggtgcttttttt

SEQ ID NO: 62 - DNA, 20 bp, Artificial Sequence
  Synthetic nucleic acid sequence
tagctgtatcgtcaaggcac

SEQ ID NO: 63 - DNA, 20 bp, Artificial Sequence
  Synthetic nucleic acid sequence
ggcctgctgaaaatgactga

SEQ ID NO: 64 - DNA, 20 bp, Artificial Sequence
  Synthetic nucleic acid sequence
  misc_feature (1)..(1): This position may be 5′ modified with HEX.
  misc_feature (9)..(10): ZEN modification may be between positions 9 and 10.
  misc_feature (12)..(12): This position may be a mutant base location.
  misc_feature (20)..(20): This position may be 3′ modified with 3IABkFQ.
tgcctacgccaccagctcca

SEQ ID NO: 65 - DNA, 20 bp, Artificial Sequence
  Synthetic nucleic acid sequence
  misc_feature (1)..(1): This position may be 5′ modified with 56-FAM
  misc_feature (9)..(10): ZEN modification may be between positions 9 and 10.
  misc_feature (12)..(12): This position may be a mutant base location.
  misc_feature (20)..(20): This position may be 3′ modified with 3IABkFQ.
tgcctacgccatcagctcca

SEQ ID NO: 66 - DNA, 23 bp, Artificial Sequence
  Synthetic nucleic acid sequence
aaacttgtggtagttggagctgg

SEQ ID NO: 67 - DNA, 23 bp, Artificial Sequence
  Synthetic nucleic acid sequence
ccagctccaactaccacaagttt

SEQ ID NO: 68 - RNA, 20 nt, Artificial Sequence
  Synthetic nucleic acid sequence
aaacuugugguaguuggagc

SEQ ID NO: 69 - DNA, 23 bp, Artificial Sequence
  Synthetic nucleic acid sequence
agtcgaggttgatggtgttcaaa

SEQ ID NO: 70 - DNA, 23 bp, Artificial Sequence
  Synthetic nucleic acid sequence
tttgaacaccatcaacctcgacc

(19) United States
(12) Patent Application Publication
(10) Pub. No.: US 2018/0051320 A1
(43) Pub. Date: Feb. 22, 2018

(54) DEPLETION OF ABUNDANT SEQUENCES BY HYBRIDIZATION (DASH)

(71) Applicant: The Regents of the University of California, Oakland, CA (US)

(72) Inventors: Joseph L. DeRisi, San Francisco, CA (US); Wei Gu, Menlo Park, CA (US); Emily D. Crawford, San Francisco, CA (US); Brian O'Donovan, San Francisco, CA (US); Michael Wilson, Austin, TX (US); Eric D. Chow, Richmond, CA (US); Hanna Retallack, San Francisco, CA (US)

(21) Appl. No.: 15/348,855

(22) Filed: Nov. 10, 2016

Related U.S. Application Data
(60) Provisional application No. 62/378,028, filed on Aug. 22, 2016.

(51) Int. Cl. C12Q 1/68

(52) U.S. Cl. CPC C12Q 1/6806; C12Q 1/6874

(57) ABSTRACT

Among other things, this disclosure describes a method comprising: cleaving a plurality of target sequences in an adaptor-tagged sequencing library using population of reprogrammed nucleic acid-directed endonucleases; non-specifically amplifying the library, thereby amplifying fragments that have not been cleaved; and sequencing the amplified sample.

### CROSS-REFERENCING

This application claims the benefit of U.S. provisional application Ser. No. 62/378,028, filed on Aug. 22, 2016, which application is incorporated by reference herein.

### BACKGROUND

The challenge of extracting faint signals from abundant noise in molecular diagnostics is a recurring theme across a broad range of applications. In the case of RNA sequencing (RNA-Seq) experiments specifically, there may be several orders of magnitude difference between the most abundant species and the least. This is especially true for metagenomic analyses of clinical samples like cerebrospinal fluid (CSF), whose source material is inherently limited, making enrichment or depletion strategies impractical or impossible to employ prior to library construction. The presence of unwanted high-abundance species, such as transcripts for the 12S and 16S mitochondrial ribosomal RNAs (rRNAs), effectively increases the cost and decreases the sensitivity of counting-based methodologies.

The same issue affects other molecular clinical diagnostics. In cancer profiling, the fraction of the mutant tumor-derived species may be vastly outnumbered by wild-type species due to the abundance of immune cells or the interspersed nature of some tumors throughout normal tissue. This problem is profoundly exaggerated in the case of cell-free DNA/RNA diagnostics, whether from malignant, transplant, or fetal sources, and relies on brute force counting by either sequencing or digital PCR (dPCR) to yield a detectable signal. For these applications, a technique to deplete specific unwanted sequences that is independent of sample preparation protocols and agnostic to measurement technology is highly desired.

Existing specific sequence enrichment techniques—such as pull-down methods, amplicon-based methods, molecular inversion methods, COLD-PCR, Competitive Allele-Specific TaqMan PCR (castPCR), and the classic method of using restriction enzyme digestion on mutant sites—can effectively enrich for targets in sequencing libraries, but these are not useful for discovery of unknown or unpredicted sequences. Brute force counting methods also exist, such as digital PCR, but they are not easy to multiplex across a large panel. While high-throughput sequencing of select regions can be highly multiplexed to detect rare and novel mutations, and barcoded unique identifiers can overcome sequencing error noise, it is costly since the vast majority of the sequencing reads map to non-informative wild-type sequences. A number of sequence-specific RNA depletion methods also currently exist. However, these methods are all employed prior to the start of library prep, and are limited to samples containing at least 10 ng to 1 μg of RNA.

Next-generation sequencing has generated a need for a broadly applicable method to remove unwanted high-abundance or wild type species prior to sequencing. The following method may meet this need.

### SUMMARY

Provided herein is a method referred to as Depletion of Abundant Sequences by Hybridization or “DASH”. Sequencing libraries can ‘DASHed’ with recombinant Cas9 protein complexed with a library of guide RNAs targeting unwanted species for cleavage, thus preventing them from consuming sequencing space. A more than 99% reduction of mitochondrial rRNA in HeLa cells has been demonstrated, as well as an enrichment of pathogen sequences in patient samples. Any application of DASH in cancer has also been demonstrated. The DASH method can be adapted for any sample type and increases sequencing yield without additional cost.

In certain embodiments, the DASH method may comprise: (a) cleaving a plurality of target sequences in an adaptor-tagged sequencing library using population of reprogrammed nucleic acid-directed endonucleases; (b) non-specifically amplifying the library after step (a), thereby amplifying fragments that have not been cleaved in step (a); and (c) sequencing the amplified sample produced by step (b). Kits for performing the method are also provided. The sequences cleaved in (a) may be expected to abundant in the library, for example.

Among other things, the DASH method may be used as a non-invasive diagnostic tool, with particular applications to low input samples, including cell-free DNA, RNA, or methylation targets in body fluids. In particular cases, the DASH method can be used to remove wild type sequence and/or sequences that are expected to be abundant in a sample, thereby allowing the identification of less abundant, mutant or unknown sequences in the sample.

Also provided are methods for analyzing, e.g., counting the number of copies of, a mutant locus. In some embodiments, this method may comprise (a) obtaining a complex nucleic acid sample that comprises both wild type copies of a genomic locus and mutant copies of the genomic locus, wherein mutant copies of the genomic locus have at least one mutation, e.g., a point mutation, relative to that wild type copies of the genomic locus; (b) specifically cleaving the wild type copies of the genomic locus using a population of reprogrammed nucleic acid-directed endonucleases; and (c) amplifying at least the mutant copies of the genomic locus. A kit for performing this method is also provided.

### BRIEF DESCRIPTION OF THE DRAWINGS

Certain aspects of the following detailed description are best understood when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures.

FIG. 1 shows (A) S. pyogenes Cas9 protein binds specifically to DNA targets that match the ‘NGG’ protospacer adjacent motif (PAM) site. Additional sequence specificity is conferred by a single guide RNA (sgRNA) with a 20 nucleotide hybridization domain. DNA double strand cleavage occurs three nucleotides upstream of the PAM site. (B) Depletion of Abundant Sequences by Hybridization (DASH) is used to target regions that are present at a disproportionately high copy number in a given next-generation sequencing library following tagmentation or flanking sequencing adaptor placement. Only non-targeted regions that have intact adaptors on both ends of the same molecule are subsequently amplified and represented in the final sequencing library.

FIG. 2 shows depletion of Abundant Sequences by Hybridization (DASH) targeting abundant mitochondrial ribosomal RNA in HeLa RNA extractions. (A) Normalized coverage plots showing alignment to the full-length human mitochondrial chromosome. Before treatment, three distinct peaks representing the 12S and 16S ribosomal subunits characteristically account for a large majority of the coverage (>60% of total mapped reads). After treatment, the peaks are virtually eliminated—with 12S and 16S signatures reduced 1000-fold to 0.055% of mapped reads. (B) Coverage plot of Cas9-targeted region with 12S and 16S gene boundaries across the top. Each red triangle represents one sgRNA target site. 54 target sites were chosen, spaced approximately 50 bp apart. (C) Scatterplot of the log of fragments per kilobase of transcript per million mapped reads (log-fpkm) values per human gene in the control vs. treated samples illustrate the significant reduction in reads mapping to the targeted 12S and 16S genes. DASH treatment results in 82 and 105-fold reductions in coverage for the 12S and 16S subunits, respectively. The slope of the regression line (red) fit to the untargeted genes indicates a 2.38-fold enrichment in reads mapped to untargeted transcripts. R-squared (R²) value of the regression line (0.979) indicates minimal off-target depletion. Between replicates, the R² coefficient between fpkm values across all genes is 0.994, indicating high reproducibility (three replicates). Notably, one gene, MT-RNR2-L12 (MT-RNR2-like pseudogene), shows significant depletion in the DASHed samples compared to the control.

FIG. 3 Normalized coverage plots of DASH-treated (orange) and untreated (blue) libraries generated from patient cerebrospinal fluid (CSF) samples with confirmed infections. Targeted mitochondrial rRNA genes (left) and representative genes for pathogen diagnosis (right) are depicted for the following: A) Patient 1, Balamuthia mandrillaris, B) Patient 2, Cryptococcus neoformans, C) Patient 3, Taenia solium. Across all cases, the DASH technique significantly reduced the coverage of human 12S and 16S genes by an average of 7.5-fold while increasing the coverage depth for pathogenic sequences by an average 5.9-fold. See Table 2 for relevant data.

FIG. 4 shows (A) DASH is used to selectively deplete one allele while keeping the other intact. An sgRNA in conjunction with Cas9 targets a wild-type KRAS sequence. However, since the G12D (c.35G>A) mutation disrupts the PAM site, Cas9 does not efficiently cleave the mutant KRAS sequence. Subsequent amplification of all alleles using flanking primers, as in the case of digital PCR, Sanger sequencing, or high-throughput sequencing is only effective for non-cleaved and mutant sites. KRAS WT sequence top strand: SEQ ID NO:66; KRAS WT sequence bottom strand: SEQ ID NO:67; sgRNA: SEQ ID NO:68; KRAS G12D sequence top strand: SEQ ID NO:69; and KRAS G12D sequence bottom strand: SEQ ID NO:70. (B) Three human genomic DNA samples with varying ratios of wild-type to mutant (G12D) KRAS were treated either with KRAS-targeted DASH, a non-human control DASH, or no DASH. Counts of intact wild-type and G12D sequences were then measured by droplet digital PCR (ddPCR). (C) Same data as in B, presented as percentage of mutant sequences detected. Inset shows fold enrichment of the percentage of mutant sequences with KRAS-targeted DASH versus no DASH. For both B and C, values and error bars are the average and standard deviation, respectively, of three independent experiments.

### Definitions

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

All references cited herein are incorporated by reference.

The term “sample” as used herein relates to a material or mixture of materials, typically, although not necessarily, in liquid form, containing one or more analytes of interest. The nucleic acid samples used herein may be complex in that they contain multiple different molecules that contain sequences. Genomic DNA and cDNA made from mRNA from a mammal (e.g., mouse or human) are types of complex samples. Complex samples may have more then 10⁴, 10⁵, 10⁶ or 10⁷ different nucleic acid molecules. A DNA target may originate from any source such as genomic DNA, cDNA (from RNA) or artificial DNA constructs. Any sample containing nucleic acid, e.g., genomic DNA made from tissue culture cells, a sample of tissue, an FFPE sample, a clinical, environmental, or other type of sample may be employed herein.

The term “nucleic acid sample,” as used herein denotes a sample containing nucleic acids. A nucleic acid sample used herein may be complex in that they contain multiple different molecules that contain sequences. Genomic DNA, RNA (and cDNA made from the same) from a mammal (e.g., mouse or human) are types of complex samples. Complex samples may have more then 10⁴, 10⁵, 10⁶ or 10⁷ different nucleic acid molecules. A target molecule may originate from any source such as genomic DNA, or an artificial DNA construct. Any sample containing nucleic acid, e.g., genomic DNA made from tissue culture cells or a sample of tissue, may be employed herein.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution and a number of different elements attached to a solid support at random positions (i.e., in no particular order). A mixture is not addressable. To illustrate by example, an array of spatially separated surface-bound polynucleotides, as is commonly known in the art, is not a mixture of surface-bound polynucleotides because the species of surface-bound polynucleotides are spatially distinct and the array is addressable.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., peptide nucleic acid or PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylenecarbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) and/or deoxyribonucleotide monomers. An oligonucleotide may be 10 to 20, 21 to 30, 31 to 40, 41 to 50, 51to 60, 61 to 70, 71 to 80, 80 to 100, 100 to 150 or 150 to 200 nucleotides in length, for example.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with their use in synthesis of primer extension products, and are usually in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18 to 40, 20 to 35, 21 to 30 nucleotides long, and any length between the stated ranges. Typical primers can be in the range of between 10 to 50 nucleotides long, such as 15 to 45, 18 to 40, 20 to 30, 21 to 25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

The term “hybridization” or “hybridizes” refers to a process in which a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions include hybridization at about 42° C. in 50% formamide, 5× SSC, 5× Denhardt's solution, 0.5% SDS and 100 ug/ml denatured carrier DNA followed by washing two times in 2× SSC and 0.5% SDS at room temperature and two additional times in 0.1×SSC and 0.5% SDS at 42° C.

The term “duplex,” or “duplexed,” as used herein, describes two complementary polynucleotides that are base-paired, i.e., hybridized together.

The term “amplifying” as used herein refers to the process of synthesizing nucleic acid molecules that are complementary to one or both strands of a template nucleic acid. Amplifying a nucleic acid molecule may include denaturing the template nucleic acid, annealing primers to the template nucleic acid at a temperature that is below the melting temperatures of the primers, and enzymatically elongating from the primers to generate an amplification product. The denaturing, annealing and elongating steps each can be performed one or more times. In certain cases, the denaturing, annealing and elongating steps are performed multiple times such that the amount of amplification product is increasing, often times exponentially, although exponential amplification is not required by the present methods. Amplification typically requires the presence of deoxyribonucleoside triphosphates, a DNA polymerase enzyme and an appropriate buffer and/or co-factors for optimal activity of the polymerase enzyme. The term “amplification product” refers to the nucleic acid sequences, which are produced from the amplifying process as defined herein.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

The term “ligating”, as used herein, refers to the enzymatically catalyzed joining of the terminal nucleotide at the 5′ end of a first DNA molecule to the terminal nucleotide at the 3′ end of a second DNA molecule.

A “plurality” contains at least 2 members. In certain cases, a plurality may have at least 2, at least 5, at least 10, at least 100, at least 100, at least 10,000, at least 100,000, at least 10⁶, at least 10⁷, at least 10⁸ or at least 10⁹ or more members.

If two nucleic acids are “complementary”, they hybridize with one another under high stringency conditions. The term “perfectly complementary” is used to describe a duplex in which each base of one of the nucleic acids base pairs with a complementary nucleotide in the other nucleic acid. In many cases, two sequences that are complementary have at least 10, e.g., at least 12 or 15 nucleotides of complementarity.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” refers to the so-called parallelized sequencing-by-synthesis or sequencing-by-ligation platforms currently employed by Illumina, Life Technologies, Pacific Biosciences and Roche etc. Next-generation sequencing methods may also include nanopore sequencing methods or electronic-detection based methods such as Ion Torrent technology commercialized by Life Technologies.

The term “extending”, as used herein, refers to the extension of a primer by the addition of nucleotides using a polymerase. If a primer that is annealed to a nucleic acid is extended, the nucleic acid acts as a template for extension reaction.

The term “barcode sequence”, “molecular barcode” or “index”, as used herein, refers to a unique sequence of nucleotides used to (a) identify and/or track the source of a polynucleotide in a reaction and/or (b) count how many times an initial molecule is sequenced (e.g., in cases where substantially every molecule in a sample is tagged with a different sequence, and then the sample is amplified). A barcode sequence may be at the 5′-end, the 3′-end or in the middle of an oligonucleotide, or both the 5′ end and the 3′ end. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179; and the like. In particular embodiments, a barcode sequence may have a length in range of from 4 to 36 nucleotides, or from 6 to 30 nucleotides, or from 8 to 20 nucleotides.

As used herein, the term “PCR reagents” refers to all reagents that are required for performing a polymerase chain reaction (PCR) on a template. As is known in the art, PCR reagents essentially include a first primer, a second primer, a thermostable polymerase, and nucleotides. Depending on the polymerase used, ions (e.g., Mg²⁺) may also be present. PCR reagents may optionally contain a template from which a target sequence can be amplified.

The term “tailed”, in the context of a tailed primer or a primer that has a 5′ tail, refers to a primer that has a region (e.g., a region of at least 12-50 nucleotides) at its 5′ end that does not hybridize to the same target as the 3′ end of the primer.

The term “target nucleic acid molecule” refers to a single molecule that may or may not be present in a composition with other target nucleic acid molecules. An isolated target nucleic acid molecule refers to a single molecule that is present in a composition that does not contain other target nucleic acid molecules.

The term “variable”, in the context of two or more nucleic acid sequences that are variable, refers to two or more nucleic acids that have different sequences of nucleotides relative to one another. In other words, if the polynucleotides of a population have a variable sequence, then the nucleotide sequence of the polynucleotide molecules of the population varies from molecule to molecule. The term “variable” is not to be read to require that every molecule in a population has a different sequence to the other molecules in a population.

The term “adaptor” refers to a nucleic acid that can be joined, via a ligase or transposon mediated reaction for example, to the ends of a double-stranded DNA molecule. As would be apparent, one end of an adaptor may be designed to be compatible with overhangs made by cleavage by an endonuclease, e.g., it may have blunt ends or a 5′ T overhang. In other embodiments, an adaptor may have a blunt end. The term “adaptor” refers to molecules that are at least partially double-stranded. An adaptor may be 10 to 150 bases in length, e.g., 50 to 120 bases, although adaptors outside of this range are envisioned.

The term “universal adaptor” refers to an adaptor that is ligated to both ends of the nucleic acid molecules under study. In certain embodiments, the universal adaptor may be a Y-adaptor. Amplification of nucleic acid molecules that have been ligated to Y-adaptors at both ends results in an asymmetrically tagged nucleic acid, i.e., a nucleic acid that has a 5′ end containing one tag sequence and a 3′ end that has another tag sequence.

The term “Y-adaptor” refers to an adaptor that contains: a double-stranded region and a single-stranded region in which the opposing sequences are not complementary. The end of the double-stranded region can be joined to target molecules such as double-stranded fragments of genomic DNA, e.g., by ligation. Each strand of an adaptor-tagged double-stranded DNA that has been ligated to a Y adaptor is asymmetrically tagged in that it has the sequence of one strand of the Y-adaptor at one end and the other strand of the Y-adaptor at the other end. Amplification of nucleic acid molecules that have been joined to Y-adaptors at both ends results in an asymmetrically tagged nucleic acid, i.e., a nucleic acid that has a 5′ end containing one tag sequence and a 3′ end that has another tag sequence.

The term “adaptor-tagged,” as used herein, refers to a nucleic acid that has been tagged by an adaptor. The adaptor can be joined to a 5′ end and/or a 3′ end of a nucleic acid molecule.

The term “tagged DNA” as used herein refers to DNA molecules that have an added adaptor sequence, i.e., a “tag” of synthetic origin. An adaptor sequence can be added (i.e., “appended”) by ligation using a ligase or via a transposase-mediated reaction.

As used herein, the term “nucleic acid guided endonuclease” refers to DNA- and RNA-guided endonucleases including the Argonaut and the Type II CRISPR/Cas-based system that is composed of two components: a nuclease (e.g., a Cas9 endonuclease or variant or ortholog thereof) that cleaves the target DNA and a guide RNA (gRNA) that targets the nuclease to a specific site in the target DNA. See, e.g., Hsu et al (Nature Biotechnology 2013 31: 827-832).

As used herein, the term, “defined site” refers to a site of known sequence.

As used herein, the term, “selectively amplifying” refers to an amplification reaction (e.g., a PCR reaction) in which only chosen sequences are amplified, e.g., using locus-specific or gene-specific PCR primers.

In certain cases, an oligonucleotide used in the method described herein may be designed using a reference genomic region, i.e., a genomic region of known nucleotide sequence, e.g., a chromosomal region whose sequence is deposited at NCBI' s Genbank database or other databases, for example. Such an oligonucleotide may be employed in an assay that uses a sample containing a test genome, where the test genome contains a binding site for the oligonucleotide.

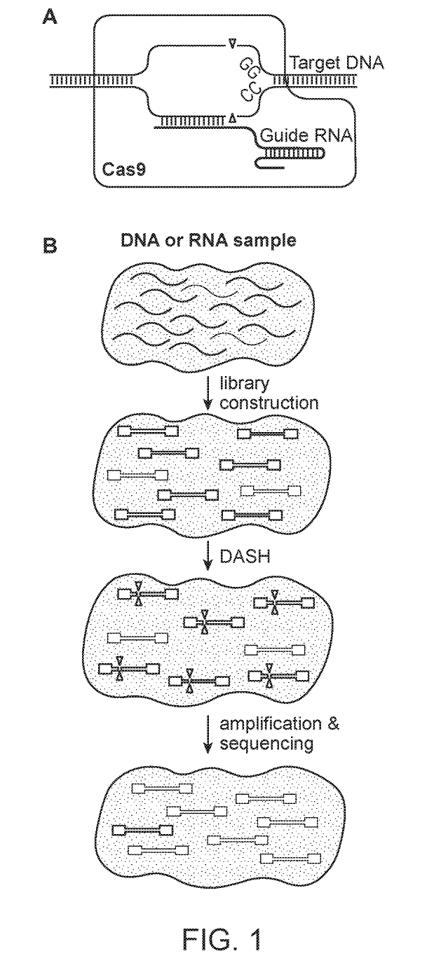

As used herein, the term, “adaptor-tagged sequencing library” refers to a library of double stranded DNA molecules that has been prepared for sequencing using a next-generation sequencing platform. Such libraries comprise double stranded DNA molecules. At least some of the molecules comprise a top strand having an added adaptor sequence at the 5′ and an added adaptor sequence at the 3′ end, and a bottom strand having an added adaptor sequence at the 5′ and an added adaptor sequence at the 3′ end. Such molecules are “asymmetrically tagged” in the sense that on any one strand the 5′ end adaptor sequence is not the same as or complementary to the 3′ adaptor sequence. In a sequencing library, all tagged molecules can usually be amplified using a single pair of primers, one that has a sequence at the 3′ end that is the same as the 3′ adaptor sequence that has been added to the library and the other that hybridizes to the 5′ adaptor sequence that has been added to the library. Apart from the adaptor sequence, all other sequence in an adaptor-tagged sequencing library may be from a natural source (e.g., a clinical sample). As will be described below, an adaptor-tagged sequence library can be made by ligating on adaptors (e.g., a Y or hairpin adaptor) to the ends of a sample comprising fragmented DNA, or by tagmentation, for example. An example of an adaptor-tagged sequencing library is shown in FIG. 1A.

If an adaptor-tagged sequencing library is “non-specifically” amplified, the library is amplified in a way that does not discriminate between the tagged molecules. This is usually done by PCR, using a pair of primers in which one of the primers hybridizes to the 5′ adaptor sequence and the other of the primers has the same sequence as the 3′ adaptor sequence.

As used herein, the term “sample that comprises both wild type copies of a genomic locus and mutant copies of the genomic locus, wherein mutant copies of the genomic locus have at least one mutation relative to that wild type copies of the genomic locus” refers to a sample that contains two alleles of a locus—a wild type allele and a mutant allele. A mutant can be generated by a substitution, insertion, deletion or inversion, for example. In many cases, the mutant copies of the locus may be in the minority relative to the wild type copies of the locus. In such a sample, the ratio of molecules that contain the wild type allele of the locus compared to molecules that contain another allele of the locus may be 1:100 or less, 1:1,000 or less, 1:10,000 or less, 1:100,000 or less or 1:1,000,000 or less.

If a method requires “specifically cleaving the wild type copies” then the cleaving step only cleaves the wild type copies of a locus, not the mutant copies. Likewise, if a guide nucleic acid targets cleavage of the wild type allele, but not the mutant allele, of a locus, then the guide nucleic acid targets cleavage of the wild type allele of the locus, not the mutant alleles of the locus.

### DETAILED DESCRIPTION

Some of the principles of the DASH method are illustrated in FIG. 1B. In some embodiments, the DASH method may comprise cleaving a plurality of target sequences in an adaptor-tagged sequencing library (where the sequencing library is double stranded and contains genomic DNA or cDNA fragments that have been tagged by “tagmentation”, addition of Y adaptors, or using tailed primers, for example) using a population of reprogrammed nucleic acid-directed endonucleases. In some cases, the target sequences may be abundant in the sample (e.g., may represent at least 0.1%, at least 0.5%, at least 1%, at least 2% or at least 5% of the total number of tagged molecules in the sample). In other cases the target sequences may guide nucleic acids target cleavage of the wild type allele, but not a mutant allele, of a locus. After the library has been cleaved, the library may be non-specifically amplified. In some cases, the adaptor-tagged sequencing library comprises strands of DNA that comprise a first adaptor sequence at the 5′ end and a second adaptor sequence at the 3′ end, and the non-specific amplification is done by PCR using primers that comprise a first primer hybridizes to the 3′ adaptor sequence and a second primer that hybridizes to the complement of the 5′ adaptor sequence. The amplification results in amplification of fragments that have not been cleaved by the endonuclease. After the library has been amplified, it is sequenced.

As would be apparent, the adaptors and/or the primers used in the method may be compatible with use in the next generation sequencing platform that is used, e.g., Illumina's reversible terminator method, Roche's pyrosequencing method (454), Life Technologies' sequencing by ligation (the SOLiD platform), Life Technologies' Ion Torrent platform or Pacific Biosciences' fluorescent base-cleavage method, etc. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) English (PLoS One. 2012 7: e47768) and Morozova (Genomics. 2008 92:255-64), which are incorporated by reference for the general descriptions of the methods and the particular steps of the methods, including all starting products, reagents, and final products for each of the steps. In some embodiments, the amplification step may be done in solution and the amplification product can be placed on a solid support (e.g., an Illumina flow cells), where the intact amplification products are amplified by bridge PCR to produce colonies. The colonies are then sequenced. In alternative embodiments, the product of the cleavage reaction can be placed directly on the solid support and amplified by bridge PCR on the support. Either way, the effect should be the same: only the uncleaved fragments will be amplified. If the amplification is done in solution, the amplification may be done using a limiting number of cycles (e.g., 4 to 20 cycles of denaturation, renaturation and extension).

The sequencing step may be done using any convenient next generation sequencing method and may result in at least 10,000, at least 50,000, at least 100,000, at least 500,000, at least 1M at least 10M at least 100M or at least 1 B sequence reads. In many cases, the reads are paired-end reads.

Depending on how the method is implemented, the endonuclease cleavage step may result in a reduction of sequence reads that would be abundant without the endonuclease cleavage step. For example, the method may result in a reduction of at least 50%, at least 80%, at least 90%, at least 95% or at least 99% of one or more sequence that would be abundant without the endonuclease cleavage step. Likewise, if the endonuclease cleavage step targets the wild type allele, but not the mutant allele, of a locus, then the number of sequence reads that correspond to the mutant copies of the locus may represent at least 1%, at least 2%, at least 5%, at least 10%, or at least 20% of the number of sequence reads that correspond to that locus.

The initial library may have been made by extracting DNA from a biological sample, and then fragmenting it (if it is not already fragmented). In these embodiments, the initial steps may be mediated by a transposase (see, e.g., Caruccio, Methods Mol. Biol. 2011; 733:241-55), in which case the fragmentation and tagging steps may be done simultaneously, i.e., in the same reaction using a process that is often referred to as “tagmentation”. In other embodiments, the fragmenting may be done mechanically (e.g., by sonication, nebulization, or shearing) or using a double stranded DNA “dsDNA” fragmentase enzyme (New England Biolabs, Ipswich MA). In some of these methods (e.g., the mechanical and fragmentase methods), after the DNA is fragmented, the ends may be polished and A-tailed prior to ligation to the adaptor. Alternatively, the ends may be polished and ligated to adaptors in a blunt-end ligation reaction. In other embodiments, the DNA in the initial sample may already be fragmented (e.g., as is the case for FPET samples and cell-free DNA (cfDNA), e.g., ctDNA, samples). The sequencing library may also contain cDNA, i.e., double-stranded DNA made from RNA. In any embodiment, the library may made from “total” nucleic acid in the sample (i.e., all the RNA, e.g., mRNA or DNA that can be extracted from the sample). Further, the DASH method can be combined with any target enrichment method, if needed. In some cases, the fragments in the sequence library may have a median size that is below 1 kb (e.g., in the range of 50 bp to 500 bp, or 80 bp to 400 bp), although fragments having a median size outside of this range may be used.

In some embodiments, the sequencing library may be made by ligating the DNA to a universal adaptor, i.e., an adaptor that ligates to both ends of the fragments of DNA in the sample. In certain cases, the universal adaptor may be added by ligating a Y adaptor (or hairpin adaptor) onto the ends of the DNA in the sample, thereby producing a double stranded DNA molecule that has a top strand that contains a 5′ tag sequence that is not the same as or complementary to the tag sequence added the 3′ end of the strand. As noted above, such a library can also be implemented by tagmentation. As should be apparent, the DNA fragments used in the initial step of the method should be non-amplified DNA that has not been denatured beforehand. In some embodiments, this step may require polishing (i.e., blunting) the ends of the cfDNA with a polymerase, A-tailing the fragments using, e.g., Taq polymerase, and ligating a T-tailed Y or hairpin adaptor to the A-tailed fragments.

The initial adaptor tagging step may be done on a limiting amount of sample (particular if the sample contains cfDNA from a bodily fluid). For example, the sample to which the adaptors are added may contain less than 200 ng of DNA, e.g., 10 pg to 200 ng, 100 pg to 200 ng, 1 ng to 200 ng or 5 ng to 50 ng, or less than 10,000 (e.g., less than 5,000, less than 1,000, less than 500, less than 100 or less than 10) haploid genome equivalents, depending on the genome. In some embodiments, the method is done using less than 50 ng of DNA (which roughly corresponds to the amount of DNA that can be obtained from approximately 5 mls of plasma) or less than 10 ng of cfDNA, which roughly corresponds to the amount of DNA that can be obtained from approximately 1 ml of plasma. In any embodiment, the adaptor may be “indexed” in that it contains a molecular barcode that identifies the sample to which it was ligated (which allows samples to be pooled before sequencing). Alternatively or in addition, the adaptor may contain a random barcode or the like. Such an adaptor can be ligated to the fragments and substantially every fragment corresponding to a particular region are tagged with a different sequence. This allows for identification of PCR duplicates and allows molecules to be counted.

In certain embodiments, the sequences targeted by the reprogrammed nucleic acid directed endonucleases may include rRNA and/or tRNA sequences although, in practice, any sequence may be targeted by the endonuclease. In one exemplary method, the sequencing library may be made from DNA or RNA of a eukaryote (e.g., a mammal), and the targeted sequences may include mitochondrial sequences (e.g., mrRNA or mtRNA sequences), because nucleic acids derived from the mitochondrial genome or transcripts from the same are often highly abundant in such samples.

In some embodiments, at least some of the target sequences are distributed throughout a target region such that, in the cleavage step, effectively all fragments from an entire region are cleaved. In these embodiments, at least some of the target sequences may occur every 30-100 bp (e.g., every 30-100 bp or 30-80 bp) over a region that is 500 bp to 20 kb (e.g., 500 bp to 5 kb) in length). In certain embodiments, the target region may include the mitochondrial MTRNR1 and/or MTRNR2 genes, which are 959 and 1559 bp in length, respectively. In some embodiments, at least 10, at least 20 or at least 30 of guide nucleic acid may contain sequences listed in Table 1, where the guide nucleic acid may also contains or may be packaged with a tracr sequence.

In embodiments in which the wild type, but not mutant, alleles of a locus are targeted by the endonucleases, the endonucleases may be targeted to sites of a mutation in any of a number of genes, including, but not limited to: ABL, AF4/HRX, AKT-2, ALK, ALK/NPM, AML1, AML1/MTG8, AXL, BCL-2, 3, 6, BCR/ABL, C-MYC, DBL, DEK/CAN, E2A/PBX1, EGFR, ENL/HRX, ERG/TLS, ERBB, ERBB-2, ETS-1, EWS/FLI-1, FMS, FOS, FPS, GLI, GSP, HER2/NEU, HOX11, HST, IL-3, INT-2, JUN, KIT, KS3, K-SAM, LBC, LCK, LMO1, LMO2, L-MYC, LYL-1, LYT-10, LYT-10/C ALPHA1, MAS, MDM-2, MLL, MOS, MTG8/AML1, MYB, MYH11/CBFB, NEU, N-MYC, OST, P53, PAX-5, PBX1/E2A, PIM-1, PRAD-1, RAF, RAR/PML, RASH, KRAS, NRAS, REL/NRG, RET, RHOM1, RHOM2, ROS, SKI, SIS, SET/CAN, SRC, TAL1, TAL2, TAN-1, TIAM1, TSC2, and TRK. Specific mutations in these genes have been correlated with a variety of disease and disorders, including breast cancer, melanoma, renal cancer, endometrial cancer, ovarian cancer, pancreatic cancer, leukemia, colorectal cancer, prostate cancer, mesothelioma, glioma, medullobastoma, polycythemia, lymphoma, sarcoma or multiple myeloma, cancers of the colon, thyroid, parathyroid, pituitary, islet cell, stomach, intestinal, embryonal, bone, renal, breast, brain, ovarian, pancreatic, uterine, eye, hair follicle, blood or uterus cancers, pilotrichomas, medulloblastomas, leiomyomas, paragangliomas, pheochromocytomas, hamartomas, gliomas, fibromas, neuromas, lymphomas and melanomas (see, e.g., (see, e.g., Chial 2008 Proto-oncogenes to oncogenes to cancer. Nature Education 1:1; Vogelstein and Kinzler 2004 Nature Medicine 10:789-799; Veltman and Brunner 2012 Nature Reviews Genetics 13:565-575).

In some embodiments, the endonucleases may be targeted to sites of a mutation in a virus, e.g., sites of mutations that make a virus drug resistant, e.g., codons 41, 62, 69, 70, 100, 101, 103, 106, 108, 181, 188, 190, 210, 215, 219, 225, 230 in the HIV-1 reverse transcriptase coding sequence, codons 10, 16, 20, 24, 32, 33, 34, 36, 46, 48, 50, 53, 54, 60, 62, 64, 71, 73, 82, 84, 85, 88, 90 and 93 in the HIV-1 protease coding sequence, codons 74, 92, 97, 121, 138, 140, 143, 148, and 155 in the HIV-1 integrase coding sequence, or codons 36, 54, 55, 155, 156, 158, 168, 170 and 175 in the HCV NS3 protease coding sequence.

For Cas9 the guide RNAs may be composed of two molecules, i.e., one RNA (“crRNA”) which hybridizes to a target and provides sequence specificity, and one RNA, the “tracrRNA”, which is capable of hybridizing to the crRNA. Alternatively, the guide RNA may be a single molecule (i.e., a sgRNA) that contains crRNA and tracrRNA sequences. A Cas9 protein may be at least 60% identical (e.g., at least 70%, at least 80%, or 90% identical, at least 95% identical or at least 98% identical or at least 99% identical) to a wild type Cas9 protein, e.g., to the Streptococcus pyogenes Cas9 protein. The Cas9 protein may have all the functions of a wild type Cas 9 protein, or only one or some of the functions, including binding activity, nuclease activity, and nuclease activity. Cas9 orthologs are known.

For Cas9 to successfully bind to DNA, the target sequence in the genomic DNA should be complementary to the gRNA sequence and must be immediately followed by the correct protospacer adjacent motif or “PAM” sequence. The PAM sequence is present in the DNA target sequence but not in the gRNA sequence. Any DNA sequence with the correct target sequence followed by the PAM sequence will be bound by Cas9. The PAM sequence varies by the species of the bacteria from which Cas9 was derived. The most widely used Type II CRISPR system is derived from S. pyogenes and the PAM sequence is NGG located on the immediate 3′ end of the gRNA recognition sequence. The PAM sequences of Type II CRISPR systems from exemplary bacterial species include: Streptococcus pyogenes (NGG), Neisseria meningitidis (NNNNGATT), Streptococcus thermophilus (NNAGAA) and Treponema denticola (NAAAAC). With some other sequence-specific nucleases, such as Argonauts, a PAM site is not required for binding and cutting the target DNA.

As would be apparent, this reaction may be done in vitro, i.e., in a cell-free environment using isolated nucleic acid (e.g., isolated DNA). The mixed sample may be collected from any source, including any organism, organic material or nucleic acid-containing substance including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In certain embodiments, the genomic DNA used in the method may be derived from a mammal, wherein certain embodiments the mammal is a human. After the endonuclease cleavage reaction has been completed, the endonuclease may be inactivated by any convenient method, e.g., using phenol chloroform or by heat denaturation. In many cases, after inactivation of the endonuclease, the nucleic acid in the sample may be purified and/or concentrated by precipitation or using a column, e.g., using Ampure beads.

The guide RNAs used in the method may be designed so that they direct binding of the endonuclease to pre-determined cleavage sites. In certain cases, the cleavage sites may be chosen so as to cleave abundant sequences, or to cleave the wild type allele of a locus, for example. Since nucleic acid isolation methods, and the nucleotide sequences of many organisms (including many bacteria, fungi, plants and animals, e.g., mammals such as human, primates, and rodents such as mouse and rat) are known, designing guide nucleic acids for use in the present method should be within the skill of one of skilled in the art. For example, Cas9-gRNA complexes can be programmed to bind to any sequence, provided that the sequence has a PAM motif. In theory, the Cas9-gRNA complexes could cleave the genomic DNA to produce fragments in the range of 30-50 bp. However, in practice, the minimal interval between the cleavage sites may be e.g., in the range of 50-80 bp. In some embodiments, the sgRNA or crRNA can be a degenerate sequence to target relatively conserved regions.

The method may make use of a set of at least 10, at least 100, at least 1,000, at least 10,000, at least 50,000 or at least 100,000 or more different guide RNAs/DNAs that are each complementary to a different, pre-defined, sites. The distance between neighboring sites may vary greatly depending on the desired application. In some embodiments, the distance between neighboring sites may be in the range of 30 bp to 150 bp, e.g., 40 bp to 100 bp.

In certain embodiments, a molar excess of endonuclease protein and guide nucleic acid may be used. For example, for Cas9, the Cas9 protein may be used in a molar excess of at least 20-fold, e.g., at least 50-fold or at least 100-fold relative to the target sequences. Likewise, for Cas9, the guide RNA may be present in a molar excess of at least 100-fold, at least 500-fold or at least 1,000-fold relative to the target sequences. Thus, each reaction may contain at least 0.1 μM Cas9 protein, e.g., at least 0.2 μM Cas9 protein, at least 0.5 μM Cas9 protein or at least 1.0 μM Cas9 protein as well as at least 1 μM sgRNA, e.g., at least 2 μM sgRNA, at least 5 μM sgRNA or at least 10 μM sgRNA.

The method described above can be employed to analyze DNA (e.g., cDNA or genomic DNA) made from virtually any organism, including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In certain embodiments, the DNA used in the method may be derived from a mammal, wherein certain embodiments the mammal is a human. In exemplary embodiments, the sample may contain genomic DNA from a mammalian cell, such as, a human, mouse, rat, or monkey cell. The sample may be made from cultured cells or cells of a clinical sample, e.g., a tissue biopsy, scrape or lavage or cells of a forensic sample (i.e., cells of a sample collected at a crime scene). In particular embodiments, the nucleic acid sample may be obtained from a biological sample such as cells, tissues, bodily fluid or excretion (e.g., stool). Bodily fluids of interest include but are not limited to, blood, serum, plasma, saliva, mucous, phlegm, cerebral spinal fluid, pleural fluid, tears, lactal duct fluid, lymph, sputum, synovial fluid, urine, amniotic fluid, and semen. In particular embodiments, a sample may be obtained from a subject, e.g., a human. In some embodiments, the sample comprises fragments of human genomic DNA. In some embodiments, the sample may be obtained from a cancer patient. In some embodiments, the sample may be made by extracting fragmented DNA from a patient sample, e.g., a formalin-fixed paraffin embedded tissue sample. In some embodiments, the patient sample may be a sample of cell-free “circulating” DNA or RNA from a bodily fluid, e.g., peripheral blood e.g. from the blood of a patient or of a pregnant female.

The method may also be applied to libraries of cloned sequences, e.g., phage, plasmis and cosmid libraries.

### Kits

Also provided by the present disclosure are kits for practicing the present method as described above. In certain embodiments, a subject kit may contain: a nucleic acid-directed endonuclease protein (e.g., Cas9); and a plurality of guide nucleic acids for the nucleic acid-directed endonuclease protein, or a template for producing the same, wherein the guide nucleic acids target cleavage of abundant sequence in a sequencing library sequence. Further details of the components of this kit are described above. The kit may also contain other reagents described above and below that may be employed in the method, depending on how the method is going to be implemented. In some embodiments, at least 10, at least 20 or at least 30 of the guide RNAs may have a sequence listed in Table 1.

In addition to above-mentioned components, the subject kit further includes instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In order to further illustrate the present invention, the following specific examples are given with the understanding that they are being offered to illustrate the present invention and should not be construed in any way as limiting its scope.

### Alternative Embodiments

The following alternative embodiments can be implemented independently from or integrated into the DASH protocol described above. These embodiments may comprise: obtaining a complex nucleic acid sample that comprises both wild type copies of a genomic locus and mutant copies of the genomic locus, wherein mutant copies of the genomic locus have at least one mutation relative to that wild type copies of the genomic locus; specifically cleaving the wild type copies of the genomic locus using a population of reprogrammed nucleic acid-directed endonucleases; and amplifying at least the mutant copies of the genomic locus. In this method, the amplifying step may comprises selectively amplifying the mutant copies of the genomic locus (e.g., using a pair of PCR primers that comprise a first primer that primers on one side of the cleavage site and a second primer that has a 3′ end that hybridizes to the nucleotide that has been mutated in the mutated copies of the locus) or by amplifying both the wild type and mutant copies of the genomic locus (e.g., using a pair of locus-specific PCR primers that comprise a first primer that primers on one side of the cleavage site and a second primer that primes on the other side of the cleavage site). Many of the reagents used in this embodiment of the methods are shared with the DASH method described in greater detail above. For example, the loci targeted by this method may be any of the loci listed above.

This method may be performed upstream of a mutation-specific assay and may be used to increase the sensitivity of such an assay by removing wild type sequences before performing the assay. In certain embodiment, the method may comprise quantifying the amount of mutant copies of the genomic locus in the sample. In these embodiments, the method may be performed upstream of a quantitative TaqMan or qInvader assay or the like. In some embodiments, the method may comprises counting the amount of mutant copies of the genomic locus in the sample. These embodiments of the method may be implemented using digital PCR, for example. In digital PCR methods, a sample is partitioned so that individual nucleic acid molecules within the sample are localized and concentrated within many separate regions. This can be done by capturing or isolating individual nucleic acid molecules has been in micro well plates, capillaries, the dispersed phase of an emulsion, and arrays of miniaturized chambers, as well as on nucleic acid binding surfaces. After partitioning, a PCR reaction is performed and the partitions have a reaction product, or a particular mutation in a reaction product, can be counted. The partitioning of the sample allows one to estimate the number of different molecules by assuming that the molecule population follows the Poisson distribution. As a result, each part will contain “0” or “1” molecules, or a negative or positive reaction, respectively. The following publications provide a detailed description of digital PCR methods: Vogelstein et al (Proc. Natl. Acad. Sci. 1999 96 (16): 9236-41); Pol et al (Expert Review of Molecular Diagnostics. Informa. 2004 4: 41-7); Dressman et al (Proc. Natl. Acad. Sci. 2003 100: 8817-22); and Pekin et al (Lab on a Chip. 2011 11: 2156-66).

Kits for performing this method may comprise a nucleic acid-directed endonuclease protein; and a guide nucleic acid for the nucleic acid-directed endonuclease protein, or a template for producing the same, wherein the guide nucleic acids target cleavage of the wild type allele, but not mutant alleles, of a locus.

### EXAMPLES

The following examples are given for the purpose of illustrating various embodiments of the invention and are not meant to limit the invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

In this example, the unique properties of Cas9 have been exploited to selectively deplete unwanted high-abundance sequences from existing RNA-Seq libraries. This approach is referred to as Depletion of Abundant Sequences by Hybridization (DASH). Employing DASH after transposon-mediated fragmentation but prior to the following amplification step (which relies on the presence of adaptor sequences on both ends of the fragment) prevents amplification of the targeted sequences, thus ensuring they are not represented in the final sequencing library (FIG. 1B). It has been shown that this technique preserves the representational integrity of the non-targeted sequences while increasing overall sensitivity in cell line samples and human metagenomic patient samples. Further, the utility of this system has been demonstrated in the context of cancer detection, in which depletion of wild-type sequences increases the detection limit for oncogenic mutant sequences. The DASH technique may be used to deplete specific unwanted sequences from existing sequencing libraries, PCR amplicon libraries, plasmid collections, phage libraries, and virtually any other existing collection of DNA species.

### Materials and Methods

Generation of cDNA from HeLa Cell Line and Clinical Samples

CSF samples were collected under the approval of the institutional review boards of the University of California San Francisco and San Francisco General Hospital. Samples were processed for high-throughput sequencing as previously described [1, 25]. Briefly, amplified cDNAs were made from randomly primed total RNA extracted from 250 μL of CSF or 250 pg of HeLa RNA using the NuGEN Ovation v.2 kit (NuGEN, San Carlos, Calif.) for low nucleic acid content samples. A Nextera protocol (Illumina, San Diego, Calif.) was used to add on a partial sequencing adapter on both sides.

In Vitro Preparation of the CRISPR/Cas9 Complex

The Cas9 expression vector, containing an N-terminal MBP tag and C-terminal mCherry, was kindly provided by Dr. Jennifer Doudna. The protein was expressed in BL21 Rosetta cells for three hours at 18° C. Cells were pelleted and frozen. Upon thawing, cells from a 4 L culture prep were resuspended in 50 mL of lysis buffer (50 mM sodium phosphate pH 6.5, 350 mM NaCl, 1 mM TCEP, 10% glycerol) supplemented with 0.5 mM EDTA, 1 μM PMSF, and a single Roche complete EDTA-free protease inhibitor tablet (Roche Diagnostics, Indianapolis, IN) and passed through an HC-8000 homogenizer (Microfluidics, Westwood, Mass.) five times. The lysate was clarified by centrifugation at 20,000 rpm for 45 minutes at 4° C. and then filtered through a 0.22 μm vacuum filtration unit. The filtered lysate was loaded onto three 5 mL HiTrap Heparin HP columns (GE Healthcare, Little Chalfont, UK) arranged in series on a GE AKTA Pure system. The columns were washed extensively with lysis buffer, and the protein was eluted with a gradient of lysis buffer to buffer B (lysis buffer supplemented with NaCl up to 1.5M). The resulting fractions were analyzed by Coomassie gel, and those containing Cas9 (centered around the point on the gradient corresponding to 750 mM NaCl) were combined and concentrated down to a volume of 1 mL using 50K MWCO Amicon Ultra-15 Centrifugal Filter Units (EMD Millipore, Billerica, Mass.) and then fed through a 0.22 μm syringe filter. Using the AKTA Pure, the 1 mL of filtered protein solution was then injected onto a HiLoad 16/600 Superdex 200 size exclusion column (GE Healthcare, Little Chalfont, UK) pre-equilibrated with buffer C (lysis buffer supplemented with NaCl up to 750 mM). Resulting fractions were again analyzed by Coomassie gel, and those containing purified Cas9 were combined, concentrated, supplemented with glycerol up to a final concentration of 50%, and frozen at −80° C. until use. Protein concentration was determined by BCA assay. Yield was approximately 80 mg from 4 L of bacterial culture.

TABLE 1
sgRNAs

| SEQ | | |
|---|---|---|
| ID | | |
| NO: | sgRNA: | Sequence: |
| 1 | mt-rRNA-1 | ATTTTCAGTGTATTGCTTTG |
| 2 | mt-rRNA-2 | ACATCACCCCATAAACAAAT |
| 3 | mt-rRNA-3 | AGGGTGAACTCACTGGAACG |
| 4 | mt-rRNA-4 | TCTAAATCACCACGATCAAA |
| 5 | mt-rRNA-5 | TTTCCCGTGGGGGTGTGGCT |
| 6 | mt-rRNA-6 | AAACTTTCGTTTATTGCTAA |
| 7 | mt-rRNA-7 | AATCGTGTGACCGCGGTGGC |
| 8 | mt-rRNA-8 | ATCTAAAACACTCTTTACGC |
| 1 | mt-rRNA-1 | ATTTTCAGTGTATTGCTTTG |
| 2 | mt-rRNA-2 | ACATCACCCCATAAACAAAT |
| 9 | mt-rRNA-9 | ACTGGAGTTTTTTACAACTC |
| 10 | mt-rRNA-10 | CACAAAATAGACTACGAAAG |
| 11 | mt-rRNA-11 | GGGGTATCTAATCCCAGTTT |
| 12 | mt-rRNA-12 | GATTTAACTGTTGAGGTTTA |
| 13 | mt-rRNA-13 | GTCCTTTGAGTTTTAAGCTG |
| 14 | mt-rRNA-14 | ACAGAACAGGCTCCTCTAGA |
| 15 | mt-rRNA-15 | TATATAGGCTGAGCAAGAGG |
| 16 | mt-rRNA-16 | TCTTCAGCAAACCCTGATGA |
| 17 | mt-rRNA-17 | CCCATTTCTTGCCACCTCAT |
| 18 | mt-rRNA-18 | TCGACCCTTAAGTTTCATAA |
| 19 | mt-rRNA-19 | TGAAACTTAAGGGTCGAAGG |
| 20 | mt-rRNA-20 | GTATACTTGAGGAGGGTGAC |
| 21 | mt-rRNA-21 | CTTTGTGTTAAGCTACACTC |
| 22 | mt-rRNA-22 | AAGGTTGTCTGGTAGTAAGG |
| 23 | mt-rRNA-23 | CATTTACCCAAATAAAGTAT |
| 24 | mt-rRNA-24 | AGTCCTTGCTATATTATGCT |
| 25 | mt-rRNA-25 | TAACTAGAAATAACTTTGCA |
| 26 | mt-rRNA-26 | CACTATTTTGCTACATAGAC |
| 27 | mt-rRNA-27 | CTACCGAGCCTGGTGATAGC |
| 28 | mt-rRNA-28 | AGGGGATTTAGAGGGTTCTG |
| 29 | mt-rRNA-29 | GGAACAGCTCTTTGGACACT |
| 30 | mt-rRNA-30 | GGCTGCTTTTAGGCCTACTA |
| 31 | mt-rRNA-31 | TTTGGGATTTTTTAGGTAGT |
| 32 | mt-rRNA-32 | GATTGGTCCAATTGGGTGTG |
| 33 | mt-rRNA-33 | ACTAACATTAGTTCTTCTAT |
| 34 | mt-rRNA-34 | TGATCTGACGCAGGCTTATG |
| 35 | mt-rRNA-35 | TGTTGGTTGATTGTAGATAT |
| 36 | mt-rRNA-36 | CTTATGAGCATGCCTGTGTT |
| 29 | mt-rRNA-29 | GGAACAGCTCTTTGGACACT |
| 30 | mt-rRNA-30 | GGCTGCTTTTAGGCCTACTA |
| 37 | mt-rRNA-37 | GAAAGGTTAAAAAAAGTAAA |
| 38 | mt-rRNA-38 | GCAGGCGGTGCCTCTAATAC |
| 39 | mt-rRNA-39 | TTTGCACGGTTAGGGTACCG |
| 40 | mt-rRNA-40 | CCTCGTGGAGCCATTCATAC |
| 41 | mt-rRNA-41 | CACGGGCAGGTCAATTTCAC |
| 42 | mt-rRNA-42 | TAATAAATTAAAGCTCCATA |
| 43 | mt-rRNA-43 | TTAGGACCTGTGGGTTTGTT |
| 44 | mt-rRNA-44 | TGCATTAAAAATTTCGGTTG |
| 45 | mt-rRNA-45 | AAGTCTTAGCATGTACTGCT |
| 46 | mt-rRNA-46 | TGTTCCGTTGGTCAAGTTAT |
| 47 | mt-rRNA-47 | GTTGATATGGACTCTAGAAT |
| 48 | mt-rRNA-48 | TACGACCTCGATGTTGGATC |
| 49 | mt-rRNA-49 | GATGGTGCAGCCGCTATTAA |
| 50 | mt-rRNA-50 | GGTCTGAACTCAGATCACGT |
| 51 | mt-rRNA-51 | TCTTGTCCTTTCGTACAGGG |
| 52 | mt-rRNA-52 | TGAGATGATATCATTTACGG |
| 53 | mt-rRNA-53 | CCCACACCCACCCAAGAACA |
| 54 | mt-rRNA-54 | ACTTAAAACTTTACAGTCAG |
| 55 | KRAS WT | AAACTTGTGGTAGTTGGAGC |
| 56 | Non-human | ACAAATATTTTAATACATGA |
| | control | |

sgRNA target sites were selected as described in the main text. DNA templates for sgRNAs based on an optimized scaffold [47] were made with a similar method to that described in [48]. See Table 1 above. For each chosen target, a 60mer oligonucleotide was purchased including the 18-base T7 transcription start site, the targeted 20mer, and the first 22 bases of the tracr RNA (5′-TAATACGACTCACTATAGNNNNNNNNNNNNNNNNNNNNGTTTAAGAGCTATGCTGG AAAC-3′) (SEQ ID NO:57). This was mixed with a 90mer representing the 3′ end of the sgRNA on the opposite strand (5′-AAAAAAAGCACCGACTCGGTGCCACTTTTTCAAGTTGATAA CGGACTAGCCTTATTTAAACTTGCTATGCTGTTTCCAGCATAGCTCTTA-3′) (SEQ ID NO:58). DNA templates for T7 sgRNA transcription were then assembled and amplified with a single PCR reaction using primers 5′-TAATACGACTCACTATAG-3′ (SEQ ID NO:59) and 5′-AAAAAAAGCACCGACTCGGTGC-3′ (SEQ ID NO:60). The resulting 131 base pair (bp) transcription templates, with the sequence 5′-TAATACGACTCACTATAGNNNNNNNNNNNN NNNNNNNNGTTTAAGAGCTATGCTGGAAACAGCATAGCAAGTTTAAATAAGGCTAG TCCGTTATCAACTTGAAAAAGTGGCACCGAGTCGGTGCTTTTTTT-3′ (SEQ ID NO:61), were pooled (for the mitochondrial rRNA library), or transcribed separately (for the KRAS experiments). All oligonucleotides were purchased from IDT (Integrated DNA Technologies, Coralville, Iowa).

Transcription was performed using custom-made T7 RNA polymerase (RNAP) [49, 50] In each 50 μL reaction, 300 ng of DNA template was mixed with T7 RNAP (final concentration 8 ng/μL), buffer (final concentrations of 40 mM Tris pH 8.0, 20 mM MgCl₂, 5 mM DTT, and 2 mM spermidine), and Ambion brand NTPs (ThermoFisher Scientific, Waltham, Mass.) (final concentration 1 mM each ATP, CTP, GTP and UTP), and incubated at 37° C. for 4 hours. Typical yields were 2-20 μg of RNA. sgRNAs were purified with a Zymo RNA Clean & Concentrator-5 kit (Zymo Research, Irvine, Calif.), aliquoted, stored at −80° C., and used only a single time after thawing.

CRISPR/Cas9 Treatment

To form the ribonucleoprotein (RNP) complex, Cas9 and the sgRNAs were mixed at the desired ratio with Cas9 buffer (final concentrations of 50 mM Tris pH 8.0, 100 mM NaCl, 10 mM MgCl₂, and 1 mM TCEP), and incubated at 37° C. for 10 minutes. This complex was then mixed with the desired amount of sample cDNA in a total of 20 μL, again in the presence of Cas9 buffer, and incubated for 2 hours at 37° C.

Since Cas9 has high nonspecific affinity for DNA [24] it was necessary to disable and remove the Cas9 before continuing. For the rRNA depletion samples, 1 μL (at >600 mAU/mL) of Proteinase K (Qiagen, Hilden, Germany) was added to each sample which was then incubated for an additional 15 minutes at 37° C. Samples were then expanded to a volume of 100 μL and purified with three phenol:chloroform:isoamyl alcohol extractions followed by one chloroform extraction in 2 mL Phase-lock Heavy tubes (5prime, Hilden, Germany). 10 μL of 3M sodium acetate pH 5.5, 3 μL of linear acrylamide and 226 μL of 100% ethanol were added to the 100 μL aqueous phase of each sample. Samples were cooled on ice for 30 minutes. DNA was then pelleted at 4° C. for 45 minutes, washed once with 70% ethanol, dried at room temperature and resuspended in 10 μL water.

In the case of the KRAS samples, Cas9 was disabled by heating the sample at 95° C. for 15 minutes in a thermocycler and then removed by purifying the sample with a Zymo DNA Clean & Concentrator-5 kit (Zymo Research, Irvine, Calif.).

High-Throughput Sequencing and Analysis of Sequencing Data

Tagmented samples with and without DASH treatment underwent 10-12 cycles of additional amplification (Kapa Amplification Kit, Kapa Biosystems, Wilmington, Mass., USA) with dual-indexing primers. A BluePippin instrument (Sage Science, Beverly, Mass., USA) was used to extract DNA between 360-540 bp. Sequencing libraries were purified using the Zymo DNA Clean & Concentrator-5 kit and amplified again on an Opticon qPCR machine (MJ Research, Waltham, Mass., USA) using a Kapa Library Amplification Kit until the exponential portion of the qPCR signal was found. Sequencing libraries were then pooled and re-quantified with a droplet digital PCR (ddPCR) Library Quantification Kit (Bio-Rad, Hercules, Calif.). Sequencing was performed on portions of one lane in an Illumina HiSeq 4000 instrument using 135 bp paired-end sequencing.

All reads were quality filtered using PriceSeqFilter v1.2 [51] such that only read pairs with less than 5 ambiguous base calls (defined as N's or positions with <95% confidence based on Phred score) were retained. Filtered reads were aligned to the hg38 build of the human genome using the STAR aligner (v 2.4.2a) [52]. The number of mapped reads per gene, and FPKM values, were calculated using the exon length and sequence information encoded in the Gencode v23 primary annotations (GTF file). Library complexity was determined by calculating the reduction in library size after clustering using the cd-hit-dup package [53, 54]. Pathogen-specific alignments to 16S and 18S sequences were accomplished using Bowtie2 [55]). Per-nucleotide coverage was calculated from alignment (SAM/BAM) files using the SAMtools suite [56] and analyzed with custom iPython [57] scripts utilizing the Pandas data package. Plots were generated with Matplotlib [58].

Digital PCR of KRAS Mutant DNA

KRAS wild-type DNA was obtained from a healthy consenting volunteer. The sample sat until cell separation occurred, and DNA was extracted from the buffy coat with the QIAamp Blood Mini Kit (Qiagen, Hilden, Germany). KRAS G12D genomic DNA from the human leukemia cell line CCRF-CEM was purchased from ATCC (Manassas, Va.). All DNA was sheared to an average of 800 bp using a Covaris M220 (Covaris, Woburn, USA) following the manufacturer's recommended settings. Cas9 reactions occurred as described above.

A primer/probe pair was designed with Primer3 [59, 60] targeting the relatively common KRAS G12D (c.35G>A) mutation. Reactions were themocycled according to manufacturer protocols using a 2-step PCR. An ideal 62° C. annealing/extension temperature was determined by a gradient experiment to ensure proper separation of FAM and HEX signals. The PCR primers and probes used were as follows (purchased from IDT): Forward: 5′-TAGCTGTATCGTCAAGGCAC-3′ (SEQ ID NO:62), Reverse: 5′-GGCCTGCTGAAAATGACTGA-3′ (SEQ ID NO:63), wild-type probe: 5′-/5HEX/TGCCTACGC/ZEN/CA<C>CAGCTCCA/3IABkFQ/-3′ (SEQ ID NO:64), mutant probe: 5′-/56-FAM/TGCCTACGC/ZEN/CA<T>CAGCTCCA/3IABkFQ/-3′ (SEQ ID NO:65), with < > denoting the mutant base location, 5HEX and 56-FAM denoting the HEX and FAM reporters, and ZEN and 3IABkFQ denoting the internal and 3′ quenchers. Original samples and those subjected to DASH were measured with the ddPCR assay on a Bio-Rad QX100 Droplet Digital PCR system (Bio-Rad, Hercules, Calif.), following the manufacturer's instructions for droplet generation, PCR amplification, and droplet reading, and using best practices. Pure CCRF-CEM samples was approximately 30% G12D and 70% wild type; all calculations of starting mixtures were made based on this starting ratio.

### Results

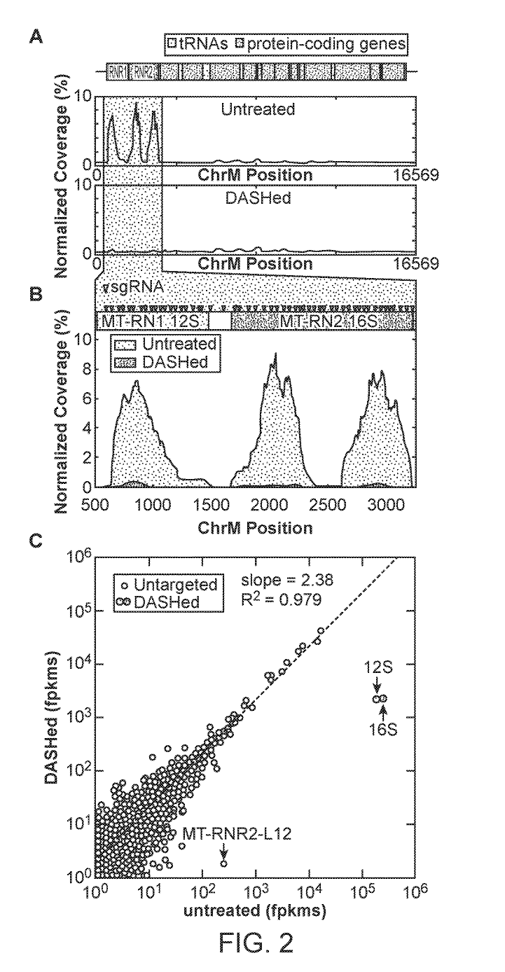

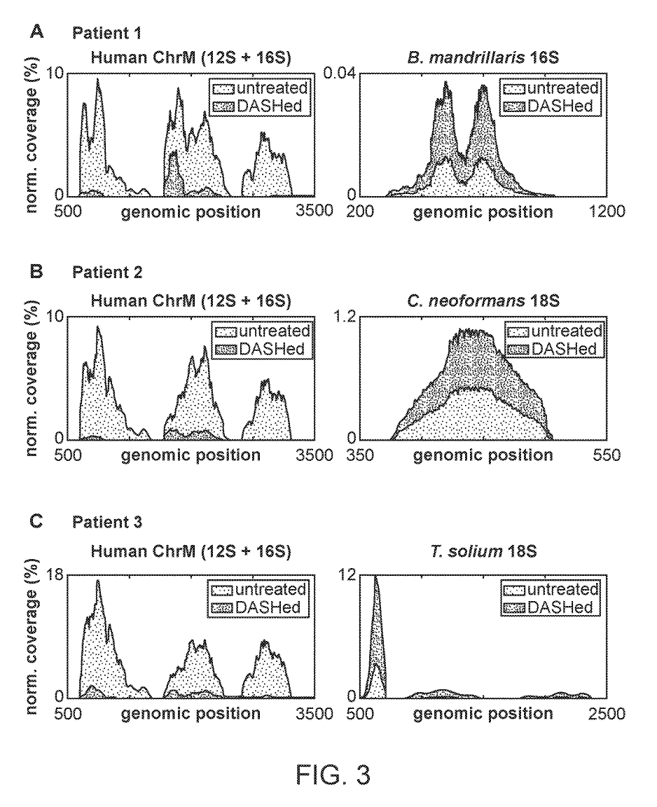

Deletion of unwanted mitochondrial ribosomal RNA using DASH was demonstrated first on HeLa cell line RNA (FIG. 2) and then on CSF RNA from patients with pathogens in their CSF (FIG. 3), in order to increase sequencing bandwidth of useful data. Selection of rRNA sgRNA targets was based on examining coverage plots for standard RNA-Seq experiments on HeLa cells as well as on several patient CSF samples. Coverage of the 12S and 16S mitochondrial rRNA genes was consistently several orders of magnitude higher than the rest of the mitochondrial and non-mitochondrial genes (FIGS. 2C and 3). Fifty-four sgRNA target sites within this high-coverage region of the mitochondrial chromosome were chosen, situated approximately every 50 bp over a 2.5 kb region (see Table 1). sgRNA sites are indicated by red arrows in FIG. 2B. sgRNAs for these sites were generated as described in the methods section.

To calculate the input ratio of Cas9 and sgRNA to sample nucleic acid, it was estimated that 90% of each sample was comprised of the rRNA regions that we targeted, thus the potential substrate makes up 4.5 ng of a 5 ng sample. This corresponds to a target site concentration of 13.8 nM in the 10 μL reaction volume. To assure the most thorough Cas9 activity possible, and given that Cas9 is a single-turnover enzyme in vitro [24], a 100-fold excess of Cas9 protein and a 1,000-fold excess of sgRNA relative to the target were used. Thus, each 10 μL sample of cDNA generated from a CSF sample contained a final concentration of 1.38 μM Cas9 protein and 13.8 μM sgRNA. In the case of HeLa cDNA, we used only 1 ng per sample, and therefore decreased the Cas9 and sgRNA concentrations by 5-fold. However, since mitochondrial rRNA sequences represented only approximately 60% of the HeLa samples (compared to approximately 90% for CSF), the HeLa samples contained 150-fold Cas9 and 1,500-fold sgRNA. To examine dose response, we processed additional 1 ng HeLa samples treated with 15-fold Cas9 and 150-fold sgRNA. Both concentrations were done in triplicate (data not shown).

Reduction of Unwanted Abundant Sequences in HeLa Samples

The utility and efficacy of DASH was first demonstrated using sequencing libraries prepared from total RNA extracted from HeLa cells. In the untreated samples, reads mapping to 12S and 16S mitochondrial rRNA genes represent 61% of all uniquely-mapped human reads. After DASH treatment, these sequences are reduced to only 0.055% of those reads (FIG. 2A and B). Comparison of gene-specific fragments per kilobase of transcript per million mapped reads (fpkm) values between treated and untreated samples reveals mean 82-fold and 105-fold decreases in fpkms for 12S and 16S rRNA, respectively, in the samples treated with 150-fold Cas9 and 1,500-fold sgRNA (FIG. 2C). Similarly, the samples treated with 15-fold Cas9 and 150-fold sgRNA show 30 and 45-fold reductions in 12S and 16S fpkm values, respectively, indicating a dose-dependent response to DASH treatment (data not shown).

Enrichment of Non-Targeted Sequences and Analysis of Off-Target Effects in HeLa Samples

This profound depletion of abundant 12S and 16S transcripts increases the available sequencing capacity for the remaining, untargeted transcripts. This increase was quantified by the slope of the regression line fit to the remaining genes, showing a 2.38-fold enrichment in fpkm values for all untreated transcripts. An R² coefficient of 0.979 for this regression line indicates strong consistency between replicates with minimal off-target effects (FIG. 2C).

To confirm that the depletion was specific to only the targeted mitochondrial sequences, the changes in fpkm values were calculated across all genes in the treated and untreated samples and identified those genes that were significantly diminished (>2 standard deviations) relative to their control values. To overcome issues with stochastic variation at low gene counts/fpkm, those genes that, between the three technical replicates at each Cas9 concentration, showed standard deviations in fpkm values greater than 50% of the mean, were eliminated. All of the genes meeting this criterion were present at less than 15 fpkm. Of the remaining genes, only one non-targeted human gene, MT-RNR2-L12, showed significant depletion when compared to the un-treated samples (FIG. 2C). MT-RNR2-L12 is a pseudogene and shares over 90% sequence identity with a portion of the 16S mitochondrial rRNA gene. Out of the 24 sgRNA sites within the homologous region, 16 of them retain intact PAM sites in MT-RNR2-L12. Of these, seven have perfectly matching 20mer sgRNA target sites, and the remaining nine each have between one and four mutations (see Supplemental FIG. 2). Depletion of this gene is therefore an expected consequence of our sgRNA choices.

Reduction of Unwanted Abundant Sequences in CSF Samples

The utility of the DASH method was applied to clinically relevant samples. In the case of pathogen detection in patient samples, the microbial transcripts are typically low in number and become greatly outnumbered by human host sequences. As a result, sequencing depth must be drastically increased to confidently detect such small minority sequence populations. It was reasoned that depletion of unwanted high-abundance sequences from patient libraries could result in increased representation of pathogen-specific sequence reads. The DASH method was integrated with an in-house metagenomic deep sequencing diagnostic pipeline for patients with meningeal inflammation (i.e., meningitis) or brain inflammation (i.e., encephalitis) likely due to an infectious agent or pathogen. FIG. 3 and Table 2 summarize the results of this analysis. In all three cases, the DASHed and untreated samples have a similar number of reads (1.8 to 3.4 million), but DASHing reduces the number of duplicate reads, indicating an increase in library complexity.

TABLE 2
Summary of depletion/enrichment results in DASH-treated clinical CSF samples.

| | | | | | | | representative | | |
|---|---|---|---|---|---|---|---|---|---|
| | read count | | targeted genes (fpkm) | | | | pathogenic gene* | | |
| | (% duplicates) | | 12S | | 16S | | (fold change) | | R² non-targeted |
| | un- | | un- | | un- | | un- | | genes, untreated |
| Pathogen | treated | DASHed | treated | DASHed | treated | DASHed | treated | DASHed | vs DASHed |
| B. mandrillaris | 1.81M | 2.54M | 298,922 | 28,005 | 380,073 | 93,164 | 0.028% | 0.102% | 0.992 |
| | (26%) | (15%) | | | | | (3.6X) | | |
| C. neoformans | 2.95M | 3.43M | 361,501 | 37,168 | 342,857 | 93,703 | 1.5% | 15.4% | 0.986 |
| | (27%) | (11%) | | | | | (10.3X) | | |
| T. solium | 2.38M | 1.89M | 451,044 | 46,993 | 317,640 | 43,257 | 12.0% | 44.3% | 0.994 |

| | | |
|---|---|---|
| (33%) | (30%) | (3.7X) |

*Representative genes are 16S for B. mandrillaris and 18S for C. neoformans and T. solium

In the case of a patient with meningoencephalitis whose CSF was previously shown to be infected with the amoeba Balamuthia mandrillaris [25] (patient 1), diagnosis was originally made by identification of a small fraction (<0.1%) of reads aligning to specific regions of the B. mandrillaris 16S mitochondrial gene. After DASH treatment, human mitochondrial 12S and 16S genes were reduced by more than an order of magnitude, and sequencing coverage of the B. mandrillaris 16S fragment increased 3.6-fold. Notably, B. mandrillaris is a eukaryotic organism, yet depletion of the human 16S gene by DASH did not have off target effects on the 16S B. mandrillaris mitochondrial gene. Similarly, patient CSF samples with confirmed Cryptococcus neoformans (fungus) (patient 2) and Taenia solium (pork tapeworm) (patient 3) infections showed 2- and 3.9-fold increases in coverage of the 18S genes of C. neoformans and, T. solium, respectively, the detection of which was crucial in the initial diagnoses. The observed increases in relative signal can be translated into either a sequencing cost savings or a higher sensitivity that may be useful clinically for earlier detection of infections.

Reduction of Wild-Type Background for Detection of the KRAS G12D (c.35G>A) Mutation in Human Cancer Samples

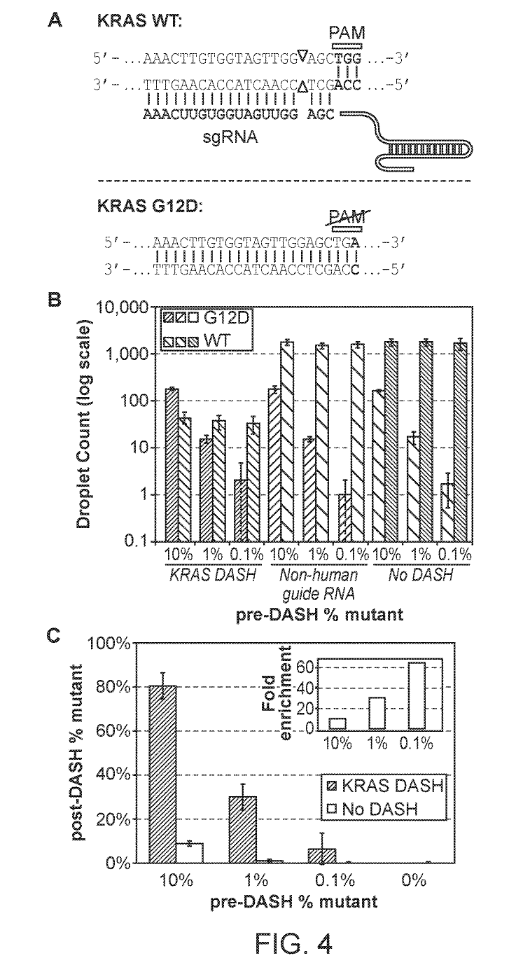

Specific driver mutations known to promote cancer evolution and at times to make up the genetic definition of malignant subtypes are important for diagnosis and targeted therapeutics (i.e. precision medicine). In complex samples isolated from biopsies or cell-free body fluids such as plasma, wild-type DNA sequences often overwhelm the signal from mutant DNA, making the application of traditional Sanger sequencing challenging [2, 3, 26]. For NGS, detection of minority alleles requires additional sequencing depth and therefore increases cost. It was reasoned that the DASH technique could be applied to increase mutation detection from a PCR amplicon derived from a patient sample. The method was used to deplete the wild-type allele of KRAS at the glycine 12 position, a hotspot of frequent driver mutations across a variety of malignancies [27-29]. This is an ideal site for DASH, because all codons encoding the wild-type glycine residue contain a PAM site (NGG), while any mutation that alters that residue (e.g., c.35G>A, p.G12D) ablates the PAM site and is thus uncleavable by Cas9 (see FIG. 4A). This will be true of any mutation that changes a glycine (codons GGA, GGC, GGG, and GGT) or a proline (codons CCA, CCC, CCG, and CCT) to any other amino acid. Furthermore, it is relevant to the ubiquitous C>T nucleotide change found in germline mutations as well as somatic cancer mutations [30]. Targeting of other mutations will likely be possible in the near future with reengineered CRISPR nucleases or those that come from alternative species and have different PAM site specificities [31, 32].

The sequence of the sgRNA designed to target the KRAS G12D PAM site is shown in Table 1 above. The sequence of non-human sequence used for the negative control sgRNA is shown in Table 1 above. Both were transcribed from a DNA template by T7 RNA polymerase, purified, and complexed with Cas9 as described in the Methods section. Samples were prepared by mixing sheared genomic DNA from a healthy individual (with wild-type KRAS genotype confirmed with digital PCR) and KRAS G12D genomic DNA to achieve mutant to wild type allelic ratios of 1:10, 1:100, and 1:1,000, and 0:1. For each mixture, 25 ng of a DNA was incubated with 25 nM Cas9 pre-complexed with 25 nM of sgRNA targeting KRAS G12D. This concentration is high relative to the concentration of target molecules, but empirically we found it to be the most efficient ratio. It was hypothesize that this may be due to non-cleaving Cas9 interactions with the rest of the human genome [24], which effectively reduce the Cas9 concentration at the cleavage site.

Samples were subsequently heated to 95° C. for 15 minutes in a thermocycler to deactivate Cas9 (Methods). Droplet digital PCR (ddPCR) was used to count wild-type and mutant alleles using the primers and TaqMan probes depicted in FIG. 4A and described in the Methods section. All samples were processed in triplicate. Samples incubated with or without Cas9 complexed to a non-human sgRNA target show the expected percentages of mutant allele: approximately 10%, 1%, and 0.1% for the 1:10, 1:100, and 1:1,000 initial mixtures respectively (FIG. 4B). With addition of Cas9 targeted to KRAS, the wild-type allele count drops nearly two orders of magnitude (purple bars in FIG. 4B), while virtually no change is observed in number of mutant alleles (blue bars). This confirms the high specificity of Cas9 for the NGG of the PAM site.

With the addition of DASH targeted to KRAS G12, the percentage of mutant allele jumps from 10% to 81%, from 1% to 30%, and from 0.1% to 6% (FIG. 4C). This corresponds to 8.1-fold, 30-fold and 60-fold representational increases for the mutant allele, respectively. As expected, there was virtually no detection of mutant alleles in the wild type-only samples both with and without DASH treatment (one droplet in one of three no DASH wild type-only samples).

The DASH method leverages the ability of Cas9 ribonucleoprotein (RNP) to deplete specific unwanted high-abundance sequences in vitro, which results in the enrichment of rare and less abundant sequences in NGS libraries or amplicon pools.

While the procedure may be easily generalized, DASH was initially developed to address current limitations in metagenomic pathogen detection and discovery, where the sequence abundance of an etiologic agent may be present as a minuscule fraction of the total. For example, infectious encephalitis is a syndrome caused by well over 100 pathogens ranging from viruses, fungi, bacteria and parasites. Because of the sheer number of diagnostic possibilities and the typically low pathogen load present in cerebrospinal fluid (CSF), more than half of encephalitis patients never have an etiologic agent identified [33]. It has been demonstrated that NGS is a powerful tool for identifying infections, but as the B. mandrillaris meningoencephalitis case demonstrates, the vast majority of sequence reads are “wasted” re-sequencing high abundance human transcripts. In this case, we have shown that DASH depletes with incredible specificity the small number of human rRNA transcripts that comprise the bulk of the NGS library, thereby lowering the required sequencing depth to detect non-human sequences and enriching the proportion of non-human (Balamuthia) reads in the metagenomic dataset. In this study, mitochondrial rRNA species were targeted because they have been consistently observed to be the most abundant sequences in these CSF-derived RNA samples. For other types of tissues, alternate programming of DASH for removal of nuclear rRNA species or essentially any other abundant sequences would he warranted.

In the case of infectious agents, it is possible to directly enrich rare sequences by hybridization to DNA microarrays [34] or beads [12]. However, these approaches rely on sequence similarity between the target and the probe and therefore may miss highly divergent or unanticipated species. Furthermore, the complexity and cost of these approaches will continue to increase with the known spectrum of possible agents or targets. In contrast, the identity and abundance of unwanted sequences in most human tissues and sample types has been well described in scores of previous transcriptome profiling projects [23], and therefore optimized collections of sgRNAs for DASH depletion are likely to remain stable.

A number of methods for depleting ribosomal RNA from RNA-Seq libraries exist in the form of commercially available kits. It is believed that DASH is equally effective or better than these methods on four metrics: (1) input requirements, (2) performance, (3) programmability, and (4) cost. These can be assessed based on information available on company websites or in publications for three major competing techniques: Illumina's Ribo-Zero and Thermo Fisher's RiboMinus, which both use biotinylated capture probes for depletion; and New England Biolab's NEBNext rRNA depletion kit, which uses RNAse H for depletion.

(1) Input Requirements: Illumina recommends 1 μg of total RNA as input for Ribo-Zero, but also has a low-input protocol requiring only 100 ng [35]. ThermoFisher recommends 2-10 μg of total RNA for its standard RiboMinus protocol [36], and 100 ng to 1 μg for its Low Input RiboMinus Eukaryote System v2 [37]. NEB recommends 10 ng-1 μg total RNA input for the NEBNext rRNA Depletion Kit [38]. The reason for these stringent amount requirements is that these three methods all deplete samples at the RNA stage. DASH, in contrast, avoids the need to delicately manipulate the original sample. Instead, DASH is employed after cDNA synthesis and library generation, thus it can be performed on any library, without regards to starting total RNA amount, or the manner in which the library was constructed (tagmentation or otherwise). For scarce and precious samples, such as patient CSF, often less than 10 ng of total cDNA is available even after NuGEN Ovation amplification; prior to this work, no commercial depletion method was available for these samples.

(2) Performance: All commercial rRNA depletion methods promise at least 85% reduction in reads of the sequences they target. Illumina states that the Ribo-Zero technique can achieve between 85% and >99% reduction in the rRNA sequences it targets [35]; RiboMinus states 95-98% reduction [39]; and NEBNext states 95-99% reduction [38]. Adiconis et al. compared several RNA-Seq methods and reported on many metrics, including depletion of rRNA sequences [23]. Ribosomal RNA sequences comprised 84.7% of reads in their un-depleted sample (100 ng total RNA from K-562 cells), while Ribo-Zero reduced this to 11.3% (an 86.7% reduction), and RNAse H reduced it to 0.1% (a 99.9% reduction). In this paper, we show that DASH decreases the mitochondrial rRNA reads in HeLa total RNA from 61% to 0.055% (99.9% reduction). Adiconis et al obtained similar numbers from 1 μg total RNA samples from formalin-fixed paraffin-embedded (FFPE) kidney tissue (78.2% and 99.9% reduction for Ribo-Zero and RNAse H, respectively) and pancreas tissue (73.0% and 99.7% reduction for Ribo-Zero and RNAse H, respectively). This is comparable to DASH reduction in three patient CSF samples (82.1%, 81.4% and 88.2% reduction). However, it is important to note again that Adiconis et al. used 1 μg total RNA from tissue samples, while the DASHed CSF samples consisted of only 5 ng of NuGEN Ovation-amplified cDNA (total RNA content in the original CSF samples was too low to accurately quantify).

Another important measure of performance is maintenance of relative abundances of non-targeted sequences, such as the human transcriptome. Correlation coefficients for samples with and without DASH treatment ranged from R²=0.979 to 0.994 in this study (see FIG. 2 and Supplemental FIG. 3), slightly higher than those found by Adiconis et al. for all methods [23].

(3) Programmability: DASH can be adapted to target any sequence containing a PAM site; construction of new sgRNAs is facile and inexpensive (see Methods section). Because it is employed after sequencing adapter addition, DASH's utility is not limited to RNA-Seq; it can be applied to any library type. Examples include ATAC-Seq libraries, in which desired nuclear DNA is contaminated with a significant amount of mitochondrial DNA sequences, and microbiome sequencing, where it may be desirable to eliminate a particularly abundant species in order to better sample the underlying diversity. Since Ribo-Zero, RiboMinus and NEBNext are all proprietary kits, they cannot easily be re-programmed by the user to target other sites.

(4) Cost: Based on current publicly available list prices of the most economical kit sizes, the per-sample costs of the kits discussed here are $82.00 (Ribo-Zero Gold Kit H/M/R, [35]), $93.67 (RiboMinus Human/Mouse Transcriptome Isolation Kit, [36]) and $45.00 (NEBNext rRNA Depletion Kit H/M/R, [38]) (all in US dollars). In contrast, we calculate the cost of DASH at less than $4 per sample when Cas9 and T7 RNA polymerase are made in-house—a very sensible solution for labs that are already spending large amounts of money on NGS. Where Cas9 production is not possible, DASH can still be carried out using commercially available Cas9 protein.

DASH may also enhance the detection of rare mutant alleles that are important for liquid biopsy cancer diagnostics. Allelic depletion with DASH increases the signal (oncogenic mutant allele) to noise (wild-type allele) by more than 60 fold when studying the KRAS hotspot mutant p.G12D. Other approaches for enriching low-abundance mutations exist, such as restriction enzyme digestion and COLD-PCR. However these methods are limited when large mutation panels are required. Here we have described a single application for DASH in cancer, but the utility of this method will be fully realized by multiplexing large panels of mutation sites, using guide RNAs and PAM sites as a way to essentially create programmable restriction enzymes that can be used in a single pool. With the rapidly growing number of oncologic therapies that target particular cancer mutations, sensitive and non-invasive techniques for cancer allele detection are increasingly relevant for optimizing patient care [26]. These same techniques are also becoming increasingly important for diagnosis of earlier stage (and generally more curable) cancers as well as the detection of cancer recurrence without needing to re-biopsy the patient [2, 14, 36, 37, 38].

The potential applications of DASH are manifold. Currently, DASH can be customized to deplete any set of defined PAM-adjacent sequences by designing specific libraries of sgRNAs. Given the popularity and promise of CRISPR technologies, we anticipate the adaptation and/or engineering of CRISPR-associated nucleases with more diverse PAM sites [31, 32, 43]. A portfolio of next-generation Cas9-like nucleases would further enable DASH to deplete large and diverse numbers of arbitrarily selected alleles across the genome without constraint. We envision that DASH will be immediately useful for the development of non-invasive diagnostic tools, with applications to low input samples or cell-free DNA, RNA, or methylation targets in body fluids [4, 6, 40, 42, 44, 45].

Many other NGS applications could also benefit from depletion of specific sequences, including hemoglobin mRNA depletion for RNA-Seq of blood samples [46] and tRNA depletion for ribosome profiling studies. Depletion of pseudogenes or otherwise homologous sequences by small but consistent differences in sequences is also theoretically possible, and may serve to remove ambiguities in clinical high-throughput sequencing. Using DASH to enrich for minority variations in microbial samples may enable early discovery of pathogen drug resistance. Similarly, the application of DASH to the analysis of cell-free DNA may augment our ability to detect early markers of drug resistance in tumors [26].

Here, we have demonstrated the broad utility of DASH to enhance molecular signals in diagnostics and its potential to serve as an adaptable tool in basic science research. While the degree of regional depletion of mitochondrial rRNA was sufficient for our application, the depletion parameters were not maximized: we used only 54 sgRNA target sites out of about 250 possible S. pyogenes Cas9 sgRNA candidates in the targeted mitochondrial region. Future studies will explore the upper limit of this system while elucidating the most effective sgRNA and CRISPR-associated nuclease selections, which will likely differ based on target and application. Irrespective, depletion of unwanted sequences by DASH is highly generalizable and may effectively lower costs and increase meaningful output across a broad range of sequence-based approaches.

### Embodiments

1. A method comprising: (a) cleaving a plurality of target sequences in an adaptor-tagged sequencing library using a population of reprogrammed nucleic acid-directed endonucleases; (b) non-specifically amplifying the library after step (a), thereby amplifying fragments that have not been cleaved in step (a); and (c) sequencing the amplified sample produced by step (b).

2. The method of embodiment 1, wherein the target sequences cleaved in step (a) are abundant in the sequence library.

3. The method of any prior embodiment, wherein the target sequences cleaved in step (a) include the wild-type, but not a mutant, allele of a locus.

4. The method of any prior embodiment, wherein the adaptor-tagged sequencing library comprises strands of DNA that comprise a first adaptor sequence at the 5′ end and a second adaptor sequence at the 3′ end, and the non-specific amplifying of step (b) is done by PCR using primers that comprise a first primer hybridizes to the 3′ adaptor sequence and a second primer that hybridizes to the complement of the 5′ adaptor sequence.

5. The method of any prior embodiment, wherein the adaptor-tagged sample comprises cDNA or genomic DNA.

6. The method of any prior embodiment, wherein the targets sequences include rRNA and/or tRNA sequences.

7. The method of any prior embodiment, wherein the sequencing library is made from a eukaryote, and the targeted sequences include mitochondrial rRNA sequences.

8. The method of any prior embodiment, wherein at least some of the target sequences are distributed throughout a target region.

9. The method of embodiment 8, wherein at least some of the target sequences occur every 30-100 bp over a 500 bp to 20 kb region.

10. The method of embodiment 8, wherein at least some of the target sequences occur every 30-80 bp over a 500 bp to 5 kb region.

11. The method of embodiment 8, wherein at least some of the target sequences are in the MTRNR1 and/or MTRNR2 genes.

12. The method of any prior embodiment, wherein the sequencing library is made from a clinical sample.

13. The method of embodiment 12, wherein the clinical sample is a bodily fluid or excretion.

14. The method of embodiment 12, wherein the sequencing library is made from cfDNA or cfRNA.

15. The method of embodiment 12, wherein the sequencing library is made from a tumor biopsy.

16. The method of any prior embodiment, wherein the endonuclease is cas9 or Argonaut, an ortholog thereof, or a variant thereof.

17. The method of any prior embodiment, wherein the sequencing library is cleaved by at least 10 reprogrammed nucleic acid-directed endonucleases.

17. A kit comprising a nucleic acid-directed endonuclease protein; and a plurality of guide nucleic acids for the nucleic acid-directed endonuclease protein, or a template for producing the same, wherein the guide nucleic acids target cleavage of abundant sequences or a wild-type, but not a mutant, allele of a locus in a sequencing library.

18. The kit of embodiment 17, wherein the endonuclease protein is Cas9, Argonaut, an ortholog thereof, or a variant thereof.

19. The kit of embodiment 17 or 18, wherein the guide nucleic acids target rRNA sequences.

20. The kit of any prior kit embodiment, wherein the guide nucleic acids target mitochondrial rRNA sequences.

21. The kit of any prior kit embodiment, wherein the guide nucleic acids target cleavage at target sequences that are distributed throughout a target region.

22. The kit of any prior kit embodiment, wherein at least some of the target sequences occur every 30-100 bp over a 500 bp to 20 kb region.

23. The kit of any prior kit embodiment, wherein the target region comprises the MTRNR1 and/or MTRNR2 genes.

25. The kit of any prior kit embodiment, wherein at least 10 of the guide nucleic acids of the kit comprise a sequence of Table 1 appended to or packaged with a tracr sequence.

25. A method comprising: (a) obtaining a complex nucleic acid sample that comprises both wild type copies of a genomic locus and mutant copies of the genomic locus, wherein mutant copies of the genomic locus have at least one mutation relative to that wild type copies of the genomic locus; (b) specifically cleaving the wild type copies of the genomic locus using a population of reprogrammed nucleic acid-directed endonucleases; and (c) amplifying at least the mutant copies of the genomic locus.

26. The method of embodiment 25, wherein the amplifying step (c) comprises selectively amplifying the mutant copies of the genomic locus.

27. The method of embodiment 25 or 26, wherein the amplifying step (c) comprises amplifying both the wild type and mutant copies of the genomic locus.

28. The method of any of embodiments 25-27, wherein the method further comprises detecting the mutant copies of the genomic locus.

29. The method of any of embodiments 25-28, wherein the method further comprising sequencing the product of step (c).

30. The method of any of embodiments 25-29, wherein the method comprises quantifying the amount of mutant copies of the genomic locus in the sample.

31. The method of any of embodiments 25-30, wherein the method comprises counting the amount of mutant copies of the genomic locus in the sample.

32. The method of embodiment 32, wherein the counting is done by digital counting.

33. A kit comprising a nucleic acid-directed endonuclease protein; and a guide nucleic acid for the nucleic acid-directed endonuclease protein, or a template for producing the same, wherein the guide nucleic acids target cleavage of the wild type allele, but not mutant alleles, of a locus.

34. The kit of embodiment 33, wherein mutant alleles of the locus are associated with a disease or condition.

35. The kit of embodiment 34, wherein the kit comprises a plurality of guide nucleic acids for the nucleic acid-directed endonuclease protein, or templates for producing the same, wherein the guide nucleic acids target cleavage of the wild type alleles, but not the mutant alleles, of one or more loci.

### REFERENCES

1. Wilsoniu et al: Actionable Diagnosis of Neuroleptospirosis by Next-Generation Sequencing. N Engl J Med 2014, 370:2408-2417.
2. Bettegowda et al Detection of Circulating Tumor DNA in Early- and Late-Stage Human Malignancies. Sci Transl Med 2014, 6:224ra24-224ra24.
3. Pan et al: Brain Tumor Mutations Detected in Cerebral Spinal Fluid. Clin Chem 2015, 61:514-522.
4. De Vlaminck et al: Circulating Cell-Free DNA Enables Noninvasive Diagnosis of Heart Transplant Rejection. Sci Transl Med 2014, 6:241ra77-241ra77.
5. Fan H et al: Non-invasive prenatal measurement of the fetal genome. Nature 2012, 487:320-324.
6. Gu et al: Noninvasive prenatal diagnosis in a fetus at risk for methylmalonic acidemia. Genet Med 2014, 16:564-567.
7. Vogelstein et al: Digital PCR. Proc Natl Acad Sci 1999, 96:9236-9241.
8. Cong L et al: Multiplex Genome Engineering Using CRISPR/Cas Systems. Science 2013, 339:819-823.
9. Doudna et al: The new frontier of genome engineering with CRISPR-Cas9. Science 2014, 346.
10. Hsu P et al: Development and Applications of CRISPR-Cas9 for Genome Engineering. Cell, 157:1262-1278.
11. Jinek et al: A Programmable Dual-RNA-Guided DNA Endonuclease in Adaptive Bacterial Immunity. Science 2012, 337:816-821.
12. Briese et al: Virome Capture Sequencing Enables Sensitive Viral Diagnosis and Comprehensive Virome Analysis. mBio 2015, 6.
13. Clark et al: Performance comparison of exome DNA sequencing technologies. Nat Biotech 2011, 29:908-914.
14. Newman et al: An ultrasensitive method for quantitating circulating tumor DNA with broad patient coverage. Nat Med 2014, 20:548-554.
15. Zou et al: Quantification of Methylated Markers with a Multiplex Methylation-Specific Technology. Clin Chem 2012, 58:375-383.
16. Akhras et al: Connector Inversion Probe Technology: A Powerful One-Primer Multiplex DNA Amplification System for Numerous Scientific Applications. PLoS ONE 2007, 2:e915.
17. Hiatt et al: Single molecule molecular inversion probes for targeted, high-accuracy detection of low-frequency variation. Genome Res 2013, 23:843-854.
18. Turner et al: Massively parallel exon capture and library-free resequencing across 16 genomes. Nat Methods 2009, 6:315-316.
19. Li J et al: Replacing PCR with COLD-PCR enriches variant DNA sequences and redefines the sensitivity of genetic testing. Nat Med 2008, 14:579-584.
20. Didelot et al: Competitive allele specific TaqMan PCR for KRAS, BRAF and EGFR mutation detection in clinical formalin fixed paraffin embedded samples. Exp Mol Pathol 2012, 92:275-280.
21. Saiki et al: Enzymatic amplification of beta-globin genomic sequences and restriction site analysis for diagnosis of sickle cell anemia. Science 1985, 230:1350-1354.
22. Kinde I et al: Detection and quantification of rare mutations with massively parallel sequencing. Proc Natl Acad Sci USA 2011, 108:9530-9535.
23. Adiconis et al: Comparative analysis of RNA sequencing methods for degraded or low-input samples. Nat Methods 2013, 10:623-629.
24. Sternberg et al: DNA interrogation by the CRISPR RNA-guided endonuclease Cas9. Nature 2014, 507:62-67.
25. Wilson et al: Diagnosing Balamuthia mandrillaris Encephalitis With Metagenomic Deep Sequencing. Ann Neurol 2015, 78:722-730.
26. Oxnard et al: Noninvasive Detection of Response and Resistance in EGFR-Mutant Lung Cancer Using Quantitative Next-Generation Genotyping of Cell-Free Plasma DNA. Clin Cancer Res 2014, 20:1698-1705.
27. Almoguera et al: Most human carcinomas of the exocrine pancreas contain mutant c-K-ras genes. Cell 1988, 53:549-554.
28. Burmer et al: Mutations in the KRAS2 oncogene during progressive stages of human colon carcinoma. Proc Natl Acad Sci U S A 1989, 86:2403-2407.
29. Tam et al: Distinct Epidermal Growth Factor Receptor and KRAS Mutation Patterns in Non-Small Cell Lung Cancer Patients with Different Tobacco Exposure and Clinicopathologic Features. Clin Cancer Res 2006, 12:1647-1653.
30. Alexandrov et al.: Signatures of mutational processes in human cancer. Nature 2013, 500:415-421.
31. Kleinstiver et al: Broadening the targeting range of Staphylococcus aureus CRISPR-Cas9 by modifying PAM recognition. Nat Biotech 2015, advance online publication.
32. Zetsche et al: Cpf1 Is a Single RNA-Guided Endonuclease of a Class 2 CRISPR-Cas System. Cell, 163:759-771.
33. Granerod et al: Challenge of the unknown A systematic review of acute encephalitis in non-outbreak situations. Neurology 2010, 75:924-932.
34. Wang et al: Viral Discovery and Sequence Recovery Using DNA Microarrays. PLoS Biol 2003, 1:e2.
35. Ribo-Zero Gold rRNA Removal I Magnetic kit for human or mouse or rat [http://www.illumina.com/products/ribo-zero-gold-rrna-removal-human-mouse-rat.html]. Accessed 5 Jan. 2016.
36. RiboMinus Human/Mouse Transcriptome Isolation Kit—Thermo Fisher Scientific [http://www.thermofisher.com/order/catalog/product/K155001]. Accessed 5 Jan. 2016.
37. Low Input RiboMinus™ Eukaryote System v2 (Pub. no. MAN0007160 Rev. 2.0) [http://tools.thermofisher.com/content/sfs/manuals/MAN0007160_RiboMinus_Eukaryote_V 2_LowInput_UG_08Jan2013.pdf]. Accessed 5 Jan. 2016.
38. NEBNext® rRNA Depletion Kit (Human/Mouse/Rat)|NEB [https://www.neb.com/products/e6310-nebnext-rrna-depletion-kit-human-mouse-rat]. Accessed 5 Jan. 2016.
39. Transcriptome enrichment without ribosomal RNA for improved microarray analysis [https://www.thermofisher.com/content/dam/LifeTech/migration/en/filelibrary/nucleic-acid-purification-analysis/pdfs.par.83981.file.dat/f-075051-ribominus-lrf.pdf]. Accessed 5 Jan. 2016.
40. Imperiale T F, Ransohoff D F, Itzkowitz S H, Levin T R, Lavin P, Lidgard G P, Ahlquist D A, Berger B M: Multitarget Stool DNA Testing for Colorectal-Cancer Screening. N Engl J Med 2014, 370:1287-1297.
41. Kinde I, Munari E, Faraj S F, Hruban R H, Schoenberg M, Bivalacqua T, Allaf M, Springer S, Wang Y, Diaz L A, Kinzler K W, Vogelstein B, Papadopoulos N, Netto G J: TERT Promoter Mutations Occur Early in Urothelial Neoplasia and are Biomarkers of Early Disease and Disease Recurrence in Urine. Cancer Res 2013, 73:7162-7167.
42. Li M, Chen W, Papadopoulos N, Goodman S, Bjerregaard N C, Laurberg S, Levin B, Juhl H, Arber N, Moinova H, Durkee K, Schmidt K, He Y, Diehl F, Velculescu V E, Zhou S, Diaz L A, Kinzler K W, Markowitz S D, Vogelstein B: Sensitive digital quantification of DNA methylation in clinical samples. Nat Biotechnol 2009, 27:858-863.
43. Ran F A, Cong L, Yan W X, Scott D A, Gootenberg J S, Kriz A J, Zetsche B, Shalem O, Wu X, Makarova K S, Koonin E, Sharp P A, Zhang F: In vivo genome editing using Staphylococcus aureus Cas9. Nature 2015, 520:186-191.
44. Koh W, Pan W, Gawad C, Fan H C, Kerchner G A, Wyss-Coray T, Blumenfeld Y J, El-Sayed Y Y, Quake S R: Noninvasive in vivo monitoring of tissue-specific global gene expression in humans. Proc Natl Acad Sci 2014, 111:7361-7366.
45. Zheng Z, Liebers M, Zhelyazkova B, Cao Y, Panditi D, Lynch K D, Chen J, Robinson H E, Shim H S, Chmielecki J, Pao W, Engelman J A, lafrate A J, Le L P: Anchored multiplex PCR for targeted next-generation sequencing. Nat Med 2014, 20:1479-1484.
46. Shin H, Shannon C P, Fishbane N, Ruan J, Zhou M, Balshaw R, Wilson-McManus J E, Ng R T, McManus B M, Tebbutt S J, for the PROOF Centre of Excellence Team: Variation in RNA-Seq Transcriptome Profiles of Peripheral Whole Blood from Healthy Individuals with and without Globin Depletion. PLoS ONE 2014, 9:e91041.
47. Chen B, Gilbert L A, Cimini B A, Schnitzbauer J, Zhang W, Li G-W, Park J, Blackburn E H, Weissman J S, Qi L S, Huang B: Dynamic Imaging of Genomic Loci in Living Human Cells by an Optimized CRISPR/Cas System. Cell 2013, 155:1479-1491.
48. Lin S, Staahl B T, Alla R K, Doudna J A: Enhanced homology-directed human genome engineering by controlled timing of CRISPR/Cas9 delivery. eLife 2015, 3:e04766.
49. Davanloo P, Rosenberg A H, Dunn J J, Studier F W: Cloning and expression of the gene for bacteriophage T7 RNA polymerase. Proc Natl Acad Sci 1984, 81:2035-2039.
50. Zawadzki V, Gross H J: Rapid and simple purification of T7 RNA polymerase. Nucleic Acids Res 1991, 19:1948.
51. Ruby J G, Bellare P, DeRisi J L: PRICE: Software for the Targeted Assembly of Components of (Meta) Genomic Sequence Data. G3 GenesGenomesGenetics 2013, 3:865-880.
52. Dobin A, Davis C A, Schlesinger F, Drenkow J, Zaleski C, Jha S, Batut P, Chaisson M, Gingeras T R: STAR: ultrafast universal RNA-seq aligner. Bioinformatics 2013, 29:15-21.
53. Fu L, Niu B, Zhu Z, Wu S, Li W: CD-HIT: accelerated for clustering the next-generation sequencing data. Bioinformatics 2012, 28:3150-3152.
54. Li W, Godzik A: Cd-hit: a fast program for clustering and comparing large sets of protein or nucleotide sequences. Bioinformatics 2006, 22:1658-1659.
55. Langmead B, Salzberg S L: Fast gapped-read alignment with Bowtie 2. Nat Methods 2012, 9:357-359.
56. Li H, Handsaker B, Wysoker A, Fennell T, Ruan J, Homer N, Marth G, Abecasis G, Durbin R, 1000 Genome Project Data Processing Subgroup: The Sequence Alignment/Map format and SAMtools. Bioinforma Oxf Engl 2009, 25:2078-2079.
57. Pérez F, Granger B E: IPython: A System for Interactive Scientific Computing. Comput Sci Eng 2007, 9:21-29.
58. Hunter J D: Matplotlib: A 2D Graphics Environment. Comput Sci Eng 2007, 9:90-95.
59. Koressaar T, Remm M: Enhancements and modifications of primer design program Primer3. Bioinformatics 2007, 23:1289-1291.
60. Untergasser A, Cutcutache I, Koressaar T, Ye J, Faircloth B C, Remm M, Rozen S G: Primer3—new capabilities and interfaces. Nucleic Acids Res 2012, 40:e115-e115.

1. A method comprising:
(a) cleaving a plurality of target sequences in an adaptor-tagged sequencing library using a population of reprogrammed nucleic acid-directed endonucleases;
(b) non-specifically amplifying the library after step (a), thereby amplifying fragments that have not been cleaved in step (a); and
(c) sequencing the amplified sample produced by step (b).

2. The method of claim 1, wherein the target sequences cleaved in step (a) are abundant in the sequence library.

3. The method of claim 1, wherein the target sequences cleaved in step (a) include the wild-type, but not a mutant, allele of a locus.

4. The method of claim 1, wherein the adaptor-tagged sequencing library comprises strands of DNA that comprise a first adaptor sequence at the 5′ end and a second adaptor sequence at the 3′ end, and the non-specific amplifying of step (b) is done by PCR using primers that comprise a first primer hybridizes to the 3′ adaptor sequence and a second primer that hybridizes to the complement of the 5′ adaptor sequence.

5. The method of claim 1, wherein the adaptor-tagged sample comprises cDNA or genomic DNA.

6. The method of claim 1, wherein the target sequences include rRNA and/or tRNA sequences.

7. The method of claim 1, wherein the sequencing library is made from a eukaryote, and the targeted sequences include mitochondrial rRNA sequences.

8. The method of claim 1, wherein at least some of the target sequences are distributed throughout a target region.

9. The method of claim 8, wherein at least some of the target sequences occur every 30 to 100 bp over a 500 bp to 20 kb region.

10. The method of claim 8, wherein at least some of the target sequences are in the MTRNR1 and/or MTRNR2 genes.

11. The method of claim 1, wherein the sequencing library is made from a clinical sample.

12. The method of claim 11, wherein the clinical sample is a bodily fluid or excretion, or a tumor biopsy.

13. The method of claim 11, wherein the sequencing library is made from cfDNA or cfRNA.

14. The method of claim 1, wherein the endonuclease is cas9 or Argonaut, an ortholog thereof, or a variant thereof.

15. The method of claim 1, wherein the sequencing library is cleaved by at least 10 reprogrammed nucleic acid-directed endonucleases.

16. A kit comprising:
a nucleic acid-directed endonuclease protein; and
a plurality of guide nucleic acids for the nucleic acid-directed endonuclease protein, or a template for producing the same, wherein the guide nucleic acids target cleavage of abundant sequences or a wild-type, but not a mutant, allele of a locus in a sequencing library.

17. A method comprising:
(a) obtaining a complex nucleic acid sample that comprises both wild type copies of a genomic locus and mutant copies of the genomic locus, wherein mutant copies of the genomic locus have at least one mutation relative to that wild type copies of the genomic locus;
(b) specifically cleaving the wild type copies of the genomic locus using a population of reprogrammed nucleic acid-directed endonucleases; and
(c) amplifying at least the mutant copies of the genomic locus.

18. The method of claim 17, wherein the method comprises determining the amount of mutant copies of the genomic locus in the sample.

19. The method of claim 18, wherein the determining is done by digital counting.

20. The method of claim 17, wherein the method further comprising sequencing the product of step (c).
